(19) United States
(12) Patent Application Publication
(10) Pub. No.: US 2005/0042688 A1
(43) Pub. Date: Feb. 24, 2005

(54) Cellular analysis

(76) Inventor: Brian Hashemi, Houston, TX (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/817,644

(22) Filed: Apr. 2, 2004

Related U.S. Application Data
(63) Continuation-in-part of application No. 10/407,262, filed on Apr. 4, 2003.

(51) Int. Cl. G01N 33/53

(52) U.S. Cl. 435/7.2

(57) ABSTRACT

The present invention relates to the analysis of cells, their cytoskeletal protein, and uses thereof. More particularly, the present invention relates to methods of analyzing cytoskeletal protein for a range of applications including, methods of measuring cellular responses and methods of identifying biomolecular signatures.

### CROSS-REFERENCE TO RELATED APPLICATIONS

This application is a Continuation-In-Part of U.S. application Ser. No. 10/407,262 filed Apr. 4, 2003, which is incorporated herein by reference in its entirety.

### GOVERNMENT INTERESTS

Portions of the disclosure herein may have been supported in part by a grant from the National Aeronautics and Space Administration, Grant No. NAG 2-1357. The United States Government may have certain rights in this application.

### FIELD

The present invention relates to the analysis of cells, their cytoskeletal protein, and uses thereof. More particularly, the present invention relates to methods of analyzing cytoskeletal protein for a range of applications including, methods of measuring cellular responses and methods of identifying biomolecular signatures.

### BACKGROUND

Cells contain an intricate network of protein filaments that extend throughout the cytoplasm called the cytoskeleton. The cytoskeleton is a highly dynamic structure that reorganizes continuously in response to various internal and external stimuli and provides cells with the ability to adopt different shapes and carry out coordinated and directed movements. The cytoskeleton plays a crucial role in signal transduction and functional responses of all human cells (Rozdzial et al., Immunity 1995, 3: 623-633; Gomez, et al. Eur. J. Immunol. 1995, 25: 2673-2678) and is involved in many other aspects of cellular function including orchestration of mechanical forces inside cells (Bunnell et al. Immunity 2001, 14: 315-329; Goebel, J. Transplant. Proc. 1999, 31: 822-824).

### BRIEF DESCRIPTION OF THE DRAWINGS

FIG. 1: shows that cellular F-actin contents are sensitive to temperature. In this example, purified T cells were incubated at the indicated temperatures (4° C., room temperature (25° C.), and 37° C., respectively) for approximately 30 minutes. Cells were then fixed at the indicated temperatures and labeled with a fluorescent F-actin probe. The relative F-actin contents were measured by flow cytometry.

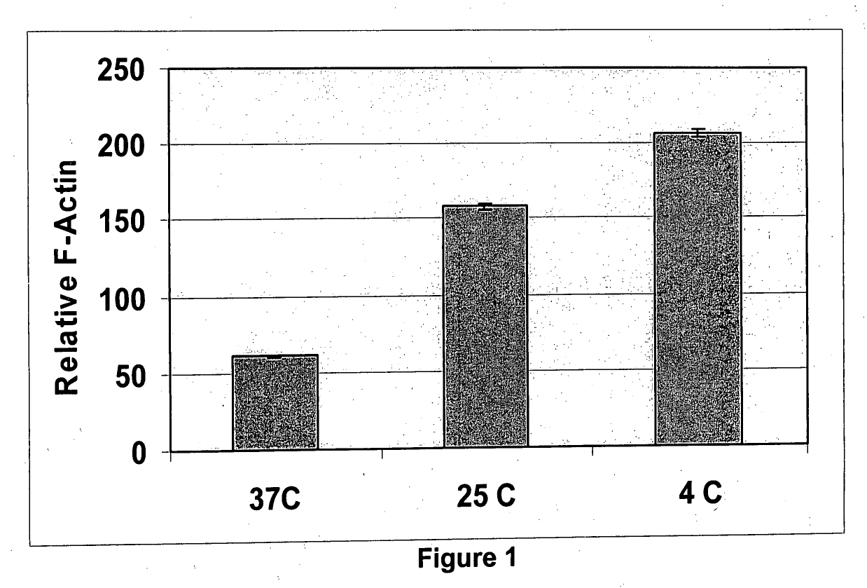

FIG. 2: shows the effect of calcium ion concentrations on the inducibility of actin polymerization in T cells. In this example, whole blood was collected from a donor in collection tubes containing heparin (curve 1) or EDTA (curve 2), respectively, as the anticoagulants. Blood samples were then incubated at 37° C. and activated with phorbol ester and a calcium ionophore (PDBu/I, phorbol-12,13-dibutyrate/ionomycin). Chelation of calcium by EDTA results in a dramatic decrease in the responsiveness of T-cells as evidenced by the lower level of inducible actin polymerization by the PDBu/I.

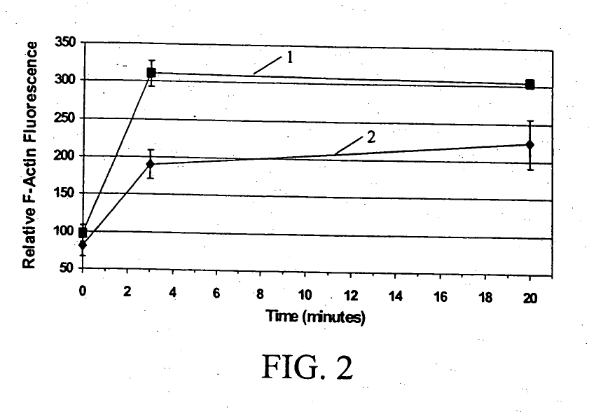

FIG. 3: shows the difference in background F-actin contents in Jurkat T cells as a function of centrifugal force and shows that activation-induced polymerization of actin is sensitive to centrifugal force and is dramatically reduced in Jurkat T cells following exposure to 300 g. RCF refers to relative centrifugal force.

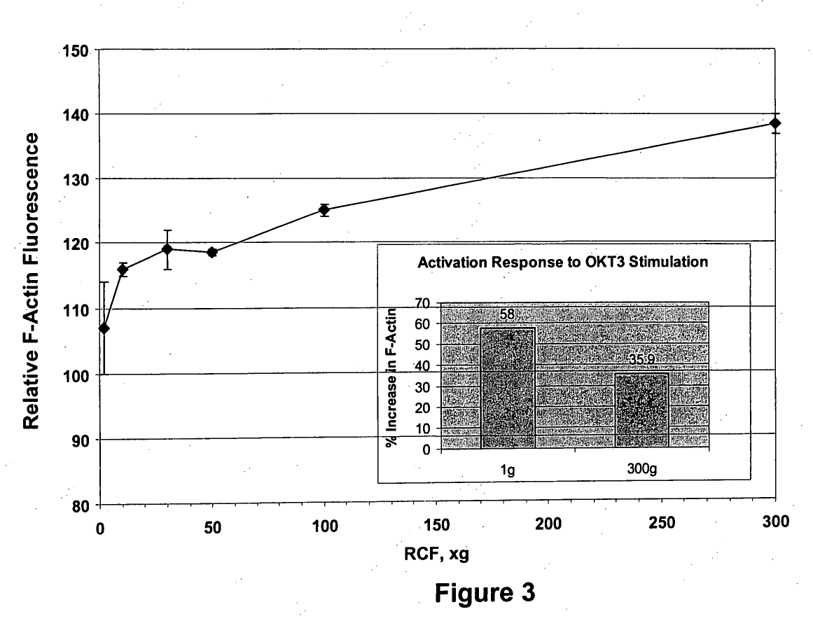

FIGS. 4A-F: Activation of whole blood cultures with an Activator Cocktail of final concentration of 30 μg/ml OKT3 for activation of T-cells, 10⁻⁶ M FMLP for activation of neutrophils, and 100 μg/ml LPS for activation of monocytes was performed for 90 seconds at 37° C. followed by fixation and labeling of F-actin by Bodippy Phallacidin and surface makers for identification of each cell type. Data were collected on a FacsCaliber instrument to assess the ability of cells to polymerize F-actin in response to receptor mediated stimulation. FIG. 4A: shows the forward scatter vs. side scatter plot and the gating used to identify neutrophils; FIG. 4B: shows the gating parameter used to identify T-cells; FIG. 4C: shows the gating parameter used to identify monocytes. FIGS. 4D-4F: shows the histogram plot for the actin content of each cell type. The relative F-actin content of each cell was measured using the actin channel and the fluorescence level of each cell is displayed on the corresponding histogram of each cell type; neutrophil F-actin (4D), T-cell F-actin (4E), and monocyte F-actin (4F). In this manner, the relative mean fluorescence associated with the F-actin content of each cell population was calculated using statistical analysis of the data.

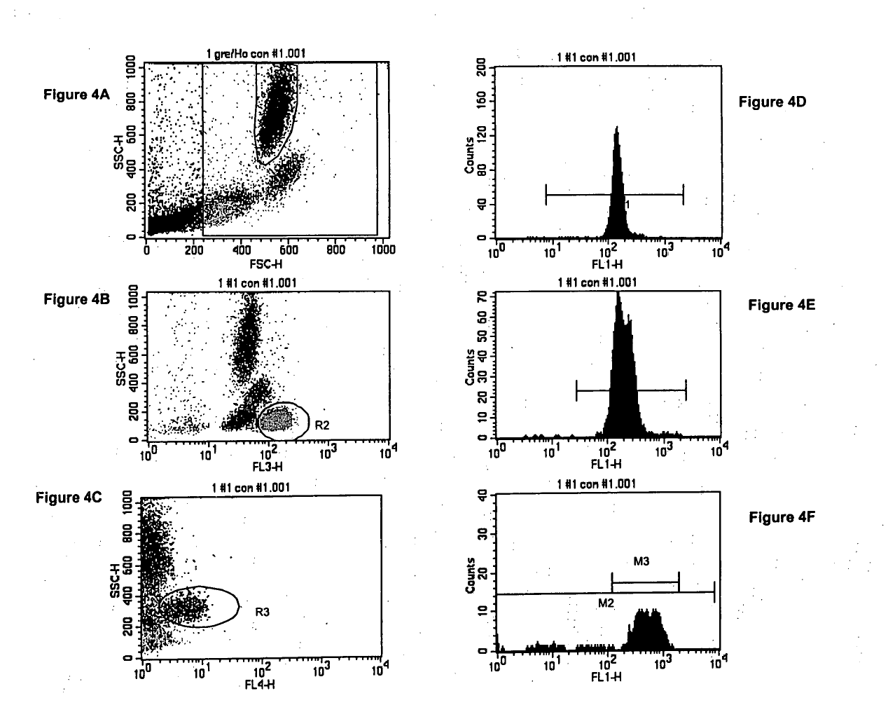

FIG. 5A-C: Dose response curves for activation of actin polymerization in whole blood samples shows the relative F-actin levels at every concentration of stimulant (NA=neutrophil F-actin; TA=T-cells F-actin; MA=monocyte F-actin). FIG. 5A: Whole blood samples were stimulated for 90 seconds with LPS. FIG. 5B: Whole blood samples were stimulated for 90 seconds with FMLP. FIG. 5C: Whole blood samples were stimulated for 90 seconds with OKT3. The error bars represent the Standard Error of Mean for duplicate samples.

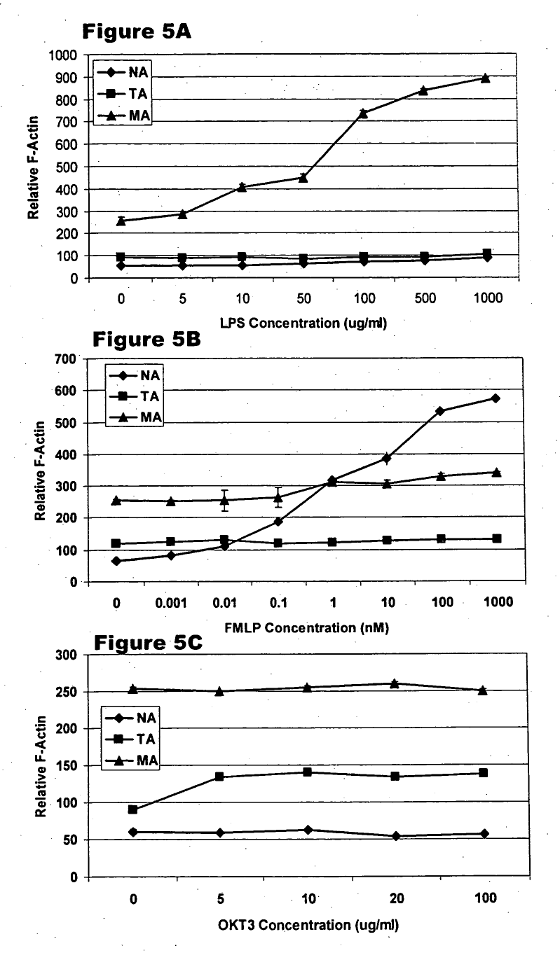

FIG. 6A: Stimulation of whole blood cultures with Activator Cocktail containing 30 μg/ml OKT3, 10⁻⁷M FMLP, and 100 μg/ml LPS at 37° C. activates polymerization of F-actin in neutrophil, monocyte, and T-cell populations. The time-course of activation indicates an optimum activation time of approximately 90 seconds for stimulation of whole blood cultures and measurement of the responsiveness of cells. FIG. 6B: F-actin levels (−) and responses to activator cocktail (+) were measured using blood samples from 6 healthy adult donors. Blood specimens were obtained at 3 time points over a two week period for all donors (total of 18 samples) and they were analyzed by the present methods.

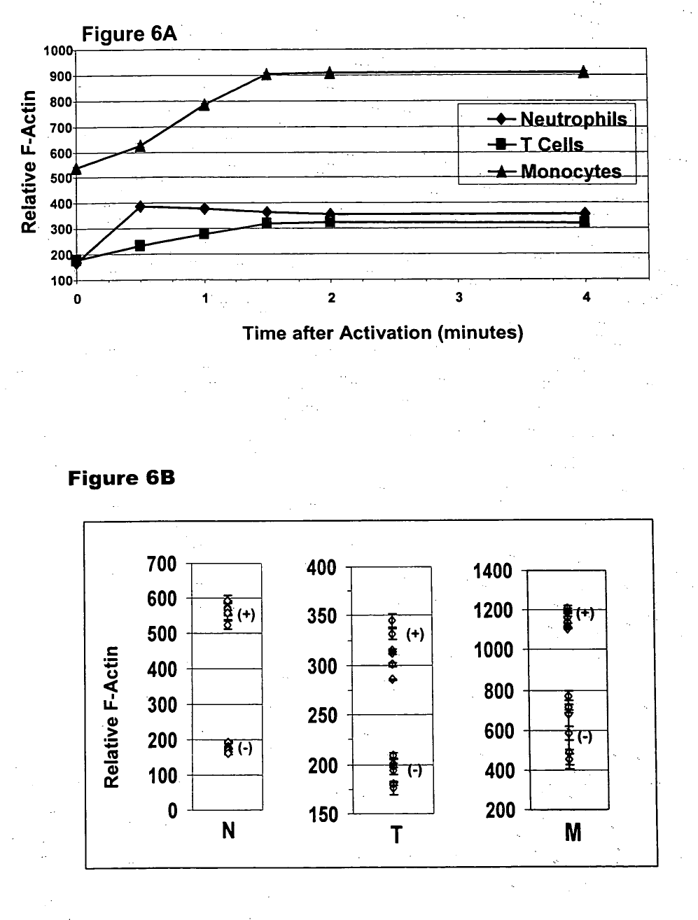

FIGS. 7A-B: 7A: Infection of whole blood cultures with live Salmonella typhimurium results in dramatic inhibition of leukocyte response to receptor-mediated activation. Blood cultures were incubated with 108 bacterial cells/ml for the indicated amount of time. Samples were then stimulated with activator cocktail containing 30 μg/ml OKT3, 10⁻⁷M FMLP, and 100 μg/ml LPS for 90 seconds at 37° C. F-actin levels were measured using the present methods and the increase in F-actin was calculated as percent activation relative to unstimulated control. (In this case the unstimulated control is blood samples that are infected with Salmonella for the same amount of time.) FIG. 7B: shows the use of cellular parameters such as population mean of F-actin, Forward Scatter, and Side Scatter for neutrophils, T-cells, and monocytes to generate a signature associated with Salmonella infection. As Salmonella infects leukocytes in whole blood cultures it imparts unique changes in the signature which is characterized by changes in signature parameters such as F-actin, Forward Scatter, and Side Scatter of neutrophils, monocytes and T-cells, as well as their responses to receptor-mediated stimulation. Salmonella infection can alter some signature parameters dramatically (up arrows) but has no effect on some of the other signature parameters (down arrows). (NA=neutrophil actin; NFS=neutrophil forward scatter; NSS=neutrophil side scatter; TA=T-cell actin; TFS=T-cell forward scatter; TSS=T-cell side scatter; MA=monocyte actin; MFS=monocyte forward scatter; MSS=monocyte side scatter. (+) indicates parameters associated with samples that were treated with activator cocktail and (−) indicates parameters associated with samples not treated with activator cocktail.)

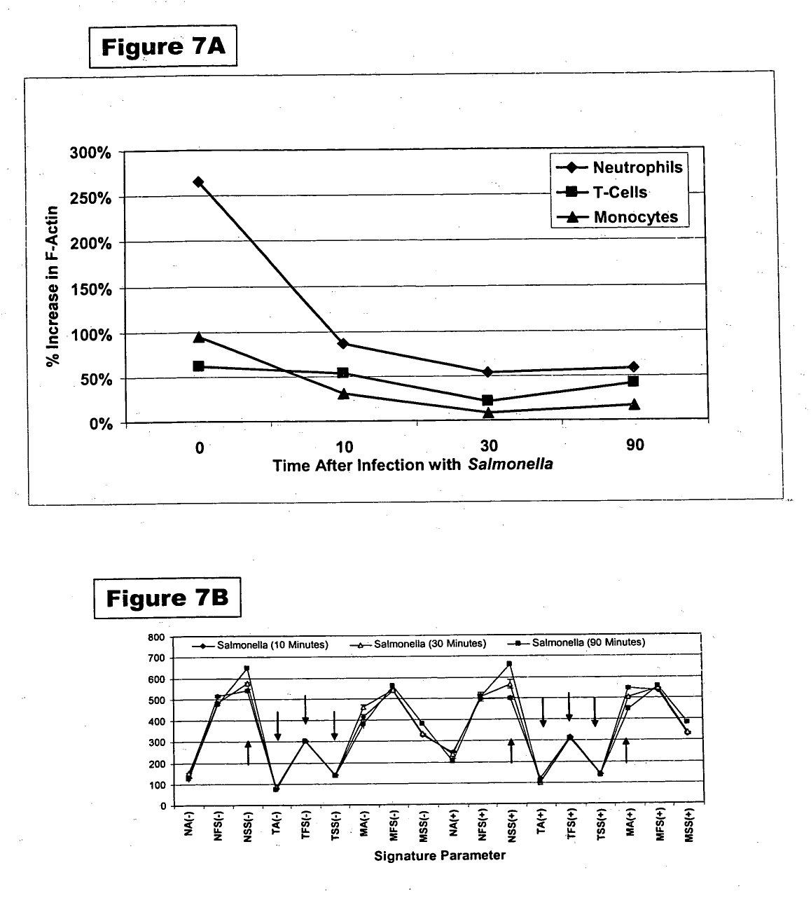

FIG. 8A-C: Biomolecular signatures of whole blood cultures infected with a variety of bacterial cells. FIG. 8A demonstrates that members of the Staphlococcus genus produce unique yet similar signatures. FIG. 8B demonstrates that gram negative Salmonella and gram positive Staphlococcus epidermis produce unique and different signatures. FIG. 8C demonstrates that two gram negative organisms from different genus Salmonella and E. Coli exhibit different signatures. (NA=neutrophil actin; NFS=neutrophil forward scatter; NSS=neutrophil side scatter; TA=T-cell actin; TFS=T-cell forward scatter; TSS=T-cell side scatter; MA=monocyte actin; MFS=monocyte forward scatter; MSS=monocyte side scatter. (+) indicates parameters associated with samples that were treated with activator cocktail for 90 seconds, and (−) indicates parameters associated with samples not treated with activator cocktail.)

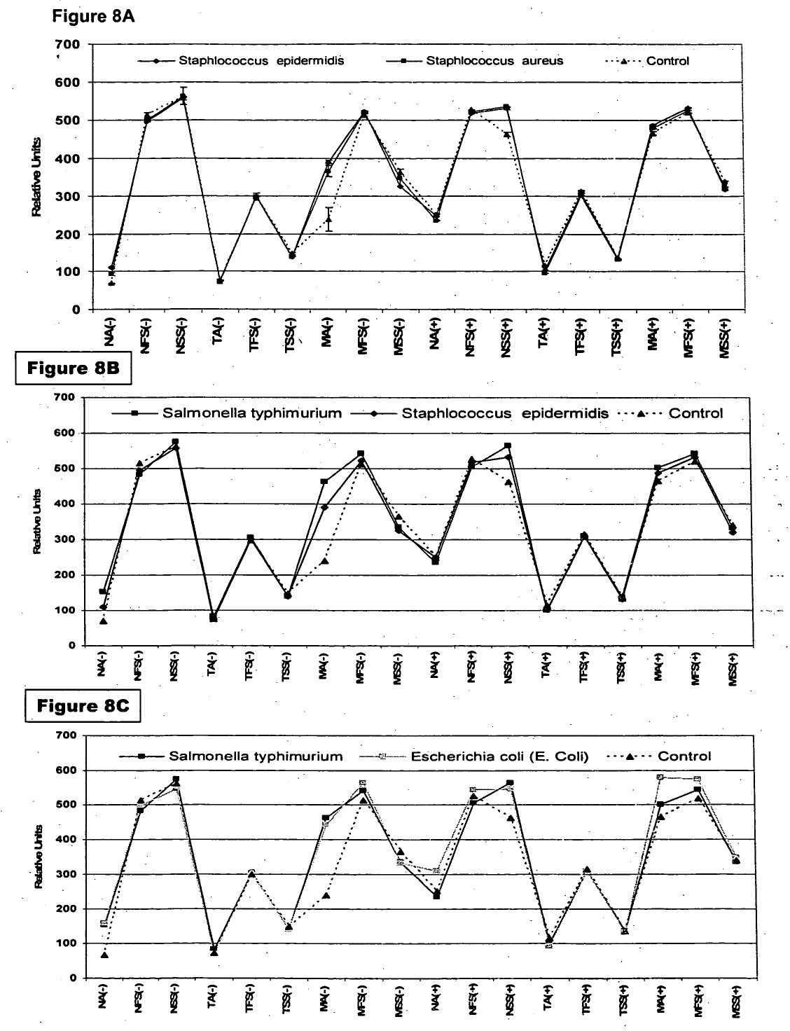

FIG. 9: FIG. 9A demonstrates the evolution of biomolecular signatures for the infection of whole blood cultures with E. Coli during the first 90 minutes of infection. FIG. 9B shows a Radargram for the signatures in 9A providing a graphical display of the unique signature of E. Coli. (NA=neutrophil actin; NFS=neutrophil forward scatter; NSS=neutrophil side scatter; TA=T-cell actin; TFS=T-cell forward scatter; TSS=T-cell side scatter; MA=monocyte actin; MFS=monocyte forward scatter; MSS=monocyte side scatter. (+) indicates parameters associated with samples that were treated with activator cocktail, and (−) indicates parameters associated with samples not treated with activator cocktail.)

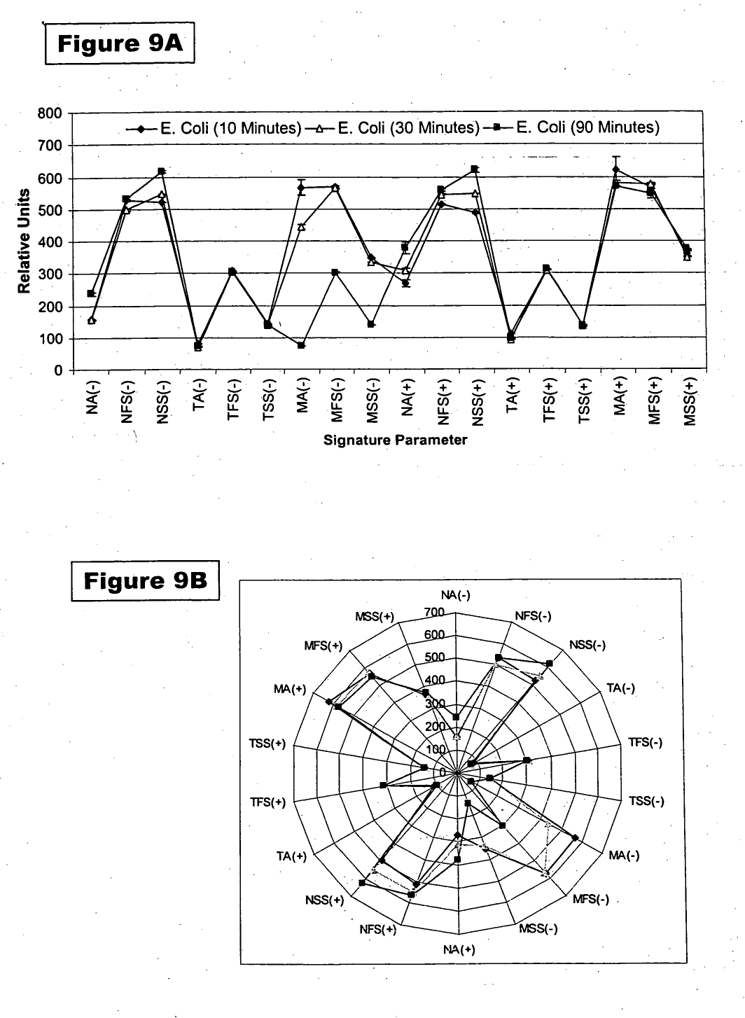

FIGS. 10A-C: FIG. 10A demonstrates the biomolecular signatures after infection of whole blood for ten minutes with select live bacteria. FIG. 10B demonstrates the biomolecular signatures after infection of whole blood for thirty minutes with select live bacteria. FIG. 10C demonstrates the biomolecular signatures after infection of whole blood for ninety minutes with select live bacteria. (NA=neutrophil actin; NFS=neutrophil forward scatter; NSS=neutrophil side scatter; TA=T-cell actin; TFS=T-cell forward scatter; TSS=T-cell side scatter; MA=monocyte actin; MFS=monocyte forward scatter; MSS=monocyte side scatter. (+) indicates parameters associated with samples that were treated with activator cocktail and (−) indicates parameters associated with samples not treated with activator cocktail.)

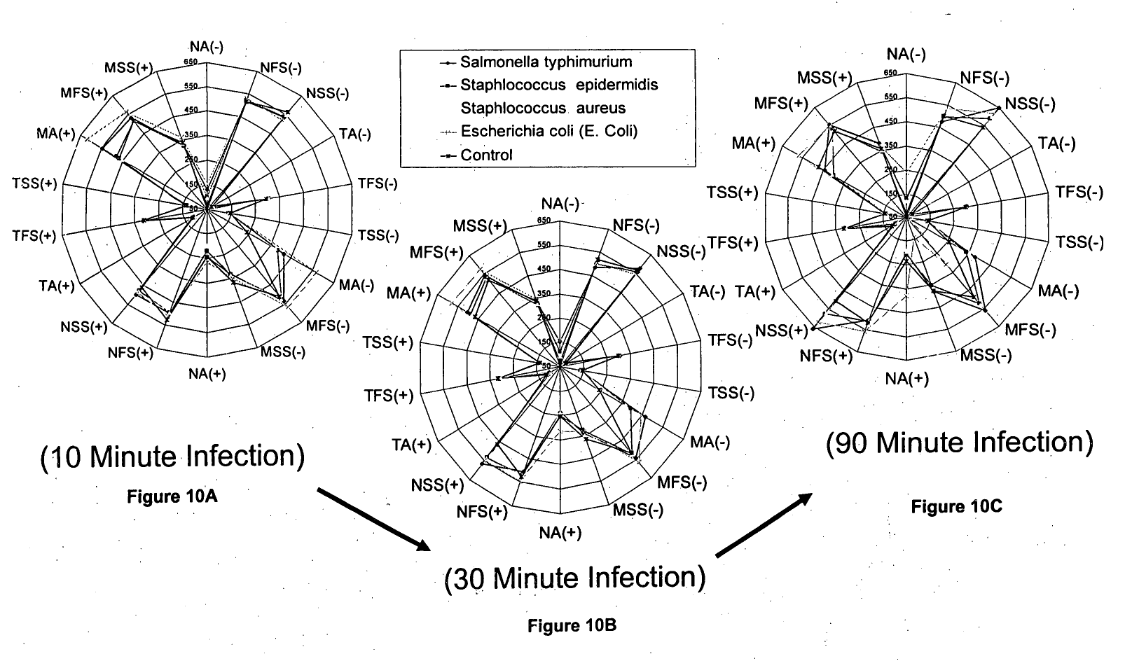

### SUMMARY

This invention relates, in part, to the discovery that by measuring changes in select biophysical properties of cells, the classification of cellular responses is made possible. Thus, the present invention provides methods of measuring changes in certain biophysical properties of cells, such as, for example, changes in the content of cytoskeletal protein in the cell, cell size, and cell granularity at different times during a cell's lifecycle and in response to a variety of biologically active agents. The present methodology permits the profiling of mammalian subjects based on the biophysical properties of their cells, and in particular, based on cellular signatures.

The present invention provides methods of identifying and using cytoskeletal signatures. As used herein, the terms “cytoskeletal signature” or “cellular cytoskeletal signature” refers to the content of cytoskeletal protein associated with a cell. Accordingly, a “cytoskeleton signature” or “cellular cytoskeleton signature” can be identified by assessing the content of cytoskeletal protein associated with one or more cell types. For use herein, cytoskeleton protein that is associated with a cell is cytoskeleton protein that is in the cell or on the surface of the cell.

The actin cytoskeleton exists in two states: monomeric or G-actin, and its polymerized state known as F-actin (fillamentous actin). Mammalian cells rely on the polymerization and de-polymerization of actin for many cellular processes. In some embodiments of the present invention, the actin cytoskeletal signature of a cell is identified. An actin signature can be identified, for example, by assessing the content of F-actin or G-actin associated with one or more cells at a certain time point.

Some embodiments of the present invention include a step of assessing the content of cytoskeletal protein associated with one or more cell types. As used herein, the term “assessing the content” can refer to determining, detecting, measuring, or quantifying the total quantity or relative quantity of one or more types of cytoskeletal protein associated with the one or more cell types. In some embodiments, “assessing the content” of cytoskeleton protein” is performed by determining the polymerization state of a certain type of cytoskeletal protein in a cell. For example, in some embodiments, “assessing the content of the cytoskeleton protein” refers to determining, detecting, measuring, or quantifying the amount of a certain type of polymerized cytoskeletal protein or unpolymerized cytoseketal protein in a cell. For example, it can refer to determining, detecting, measuring, or quantifying the amount of F-actin or G-actin in a cell.

There are many techniques for measuring cytoskeletal protein associated with a cell. All of these techniques can be used in accordance with the present invention. In accordance with some particular embodiments, the present invention provides methods for measuring cellular responses in a subject or measuring the cytoskeletal content associated with a cell comprising (i) stabilizing a mixture of cells, (ii) labeling one or more cell types from the mixture using cell type-specific reagent, and (iii) assessing the content of cytoskeletal protein associated with the one or more cell types. The present invention also provides methods for measuring cellular responses in a subject or measuring the cytoskeletal content associated with a cell that do not require the step of labeling one or more cell types from the mixture. In one aspect, the cells are stabilized at a temperature of from about 27 degrees Celsius to about 50 degrees Celsius, with a temperature of from about 30 to about 40 degrees Celsius, and in particular, a physiological temperature, i.e., a temperature of about 37 degrees Celsius, being preferred for some uses. Additional temperatures, for example, temperatures from about 4 degrees Celsius to about 50 degrees, or 25 degrees Celsius to about 40 degrees are expressly included within the scope of the present invention.

Any method of stabilizing cells can be used in accordance with the present invention. For example, the cells can be stabilized by fixation. In some aspects of the present invention, the cells are collected from a subject using a non-chelating coagulant.

Cytoskeletal protein can be assessed using any known technique to detect and/or measure cytoskeletal protein content including cytoskeleton polymerization states. For example, in accordance with some particular embodiments, the cytoskeletal protein is first labeled and microscopy techniques, such as fluorescence microscopy techniques, or flow cytometry techniques are used to assess cytoskeletal protein content. It is not always necessary to label the cytoskeletal protein before assessing the cytoskeletal protein content.

For the purposes of the present application, the term “cytoskeletal protein” or “cellular cytoskeletal protein” refers to any subset of a cytoskeletal protein, for example, cytoskeletal protein can refer to F-actin, G-actin, or total actin. The cytoskeletal protein that is assessed (e.g., detected, quantified, or measured) can be any cytoskeletal protein type including, for example, actin microfilaments, intermediate filaments, microtubules, spectrin, talin, vinculin, desmin, senaptin, vimentin, ezrin, moesin, filamin, phakinin, actinin, profilin, fibrin, keratin, myosin, dynein, and kinesin. In some embodiments, only one type of cytoskeletal protein type is assessed, e.g., only F-actin or only G-actin. In other embodiments, more than one type of cytoskeletal protein can be assessed e.g., F-actin and senaptin.

The present invention includes methods for identifying the cytoskeletal signature of a cytoskeletal protein comprising a step of assessing the content of cytoskeletal protein in or on the surface of one cell type or a plurality of cell types. In some embodiments, the methods further comprise a step of comparing the content of cytoskeletal protein associated with the one cell type or plurality of cell types to the content of corresponding cytoskeletal protein associated with corresponding cell types from a control. By assessing the content of cytoskeletal protein associated with a cell using the methods described herein, it is possible, inter alia, to determine the polymerization state of cytoskeletal protein in a cell at a certain time point.

In various embodiments of the present invention, one will be comparing the content of cytoskeletal protein and/or other cellular parameters associated with one cell type or a plurality of cell types to the content of corresponding cytoskeletal protein and/or other cellular parameters associated with corresponding cell types from a control. In other words, one will be comparing cytoskeletal signatures or biomolecular signatures in one sample comprising a mixture of cells to another sample comprising a mixture of cells. For purposes of this application, when two signatures are being compared, one signature can act as a control for another. For example, in comparing infection by a strain of Salmonella to infection by a strain of E. Coli, one of the signatures can act as a control for the other for the purposes of this application. Other examples include comparing the cytoskeletal or biomolecular signature of a blood sample from a patient with the cytoskeletal or biomolecular signature of blood samples from a healthy donor, or a group of healthy donors, in which case the healthy donor signatures serve as a control for the signature of the patient blood sample. In another example, blood samples from a patient can be exposed to a number of different drugs to compare the cytoskeletal or biomolecular signature of the patient blood sample after exposure to the drug. In this example, the biomolecular signature or cytoskeletal signature of one drug acts as a control for comparison with the biomolecular signature or cytoskeletal signature of another drug.

In some embodiments of the present invention, it will be desirable to measure not only cytoskeleton signature but additional cellular properties or parameters including, for example, cell size, cell granularity, number of receptors on the surface of a cell, number of cells in a biological sample, uptake of specific dye such as lipids dyes or nucleic acid dyes, and the like. The term “biomolecular signature” as used herein refers to the cytoskeleton signature in a cell as well as one or more additional cellular parameters.

All living creatures are made of cells. Eukaryotic cells contain a large quantity of DNA and an array of internal membranes. The cellular cytoskeleton helps organize the cell by keeping internal cellular structures in their proper place and controlling their movements. The cytoskeleton is comprised of a networks of actin microfilaments, intermediate filaments, microtubules and their related proteins. The polymerization and de-polymerization of cytoskeletal proteins is said to drive many of the cellular processes in human cells. The present inventor has discovered that by assessing the cytoskeletal protein associated with a cell, it is possible to measure and/or classify cellular responses. Methods for classifying and/or measuring cellular responses are, accordingly, encompassed by the present invention. In accordance with some particular embodiments, these methods comprise (i) assessing the content of cytoskeletal protein associated with one cell type or a plurality of cell types and (ii) comparing the content of the cytoskeletal protein associated with the one cell type or plurality of cell types to the content of corresponding cytoskeletal protein associated with corresponding cell types from a control.

Many human disorders are associated with abnormalities in the cytoskeletal proteins. For example aggregates of neurofilament proteins and aberrant accumulation of neurofilaments in motor neuron cell bodies are associated with several neurological disorders including amyotrophic lateral sclerosis, infantile spinal muscular atrophy, and hereditary sensory motor neuropathy. Neutrophil actin dysfunction has long been recognized as a cause for poor neutrophil motility, adherence, and phagocytosis in an infant with life-threatening infections (Boxer et al., N. Engl. J. Med. 1974, 291: 1093-1099). Studies have revealed an inherited genetic alteration as the cause of leukocyte actin dysfunction in some cases and an acquired leukocyte actin dysfunction in many other clinical conditions (Englich et al., Clin. Infect. Dis. 2001, 33: 2040-2048.).

Cytoskeletal proteins in or on the surface of a cell are affected by exposure to both endogenous and exogenous biologically-active agents. The present inventor has discovered that the cytoskeletal signature of cells are in flux and that cells display a unique cytoskeletal signature depending on their internal and external surroundings. Moreover, it has been discovered that cytoskeletal signatures can provide information regarding the status of a cell. For example, a cell that has been exposed to a certain species of a gram negative bacteria will present a unique cytoskeletal signature that is indicative of the cell's exposure to that certain species of gram negative bacteria. Similarly, a cell that is cancerous will present a unique cytoskeletal signature that is indicative of the cell's cancerous state. By recognizing the unique cytoskeletal signatures within a cell, it is possible to, among other things, assess the presence or absence of disease states, determine cellular response patterns to different biologically active agents such as drugs, monitor the progression of disease states in a subject, and determine donor-recipient compatibility for transplant therapy. Accordingly, the present invention provides methods for assessing the presence or absence of a disease state in a subject comprising (i) assessing the content of cytoskeletal protein associated with one cell type or a plurality of cell types from the subject and (ii) correlating the content with the presence or absence of a disease state in the subject. The present invention also provides methods for determining a response profile to a drug comprising (i) assessing the content of cytoskeletal protein associated with one cell type or a plurality of cell types from the subject and (ii) correlating the content of cytoskeletal protein with a probability of being a positive-responder, negative-responder, or non-responder to therapy with the drug.

For use herein, a positive responder, is a subject who positively responds to treatment, i.e., a subject who experiences success in amelioration of an injury, pathology, or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. A positive responder is one in which any toxic or detrimental side effects of the biologically active agent is outweighed in clinical terms by therapeutically beneficial effects. In contrast, a negative responder is one in which the therapeutically beneficial effects of the treatment is outweighed by the toxic or detrimental side effects of the biologically active agent. A non-responder is a subject who doesn't respond to the treatment or doesn't respond to a satisfactory level.

The present invention also provides methods for monitoring the progression of a disease state comprising (i) assessing the content of cytoskeletal protein associated with one cell type or a plurality of cell types from the subject and (ii) correlating the content of cytoskeletal protein with progression of the disease state in the subject.

Methods of determining donor-recipient compatibility for transplant therapy are also encompassed by the present invention. For example, a blood sample from a recipient will be exposed to a tissue from the donor and the cytoskeletal or biomolecular signature of the blood sample will be used to predict likelihood of rejection or acceptance. In another example, the cytoskeletal or biomolecular signature of a blood sample from a recipient after transplant operation will be used to assess the rejection or acceptance status of the patient. In this example the methods comprise the steps of (i) assessing the content of cytoskeletal protein associated with one cell type or a plurality of cell types from the recipient and (ii) correlating the content of cytoskeletal protein with compatibility to the transplant. Comparison of signatures from the recipient to signatures from other patients that have experienced rejection of a transplant will enable early detection of rejection in the recipient. In this example, signatures from patients who have experienced a rejection can serve as a control for the signature of the recipient.

Any mammalian cell type can be used in the present invention. The cells can be selected from a variety of tissue types including, for example, hematopoietic cells, stem cells, hepatic cells, muscle cells, nerve cells, mesenchymal cells, cartilage and/or bone cells, intestinal cells, pancreatic cells or kidney cells. Cell types include, for example, common lymphoid progenitor cells, T cells (e.g., helper, cytotoxic, and suppressor cells), B cells, plasma cells, natural killer cells, common myeloid-progenitor cells, monocytes, macrophages, mast cells, leukocytes, basophils, neutrophils, eosinophils, megakaryocytes, erythrocytes, and cell fragments such as platelets. The term “stem cell” refers to an undifferentiated cell which is capable of self-renewal, i.e., proliferation to give rise to more stem cells, and may give rise to lineage committed progenitors which are capable of differentiation and expansion into a specific lineage. As used herein, the term “stem cells” refers generally to embryonic, hematopoietic and other stem cells of mammalian, e.g., human, origin.

In one preferred embodiment, the cell will be any cell of the blood and immune system, e.g., erythrocytes, megakaryocytes, macrophages and related cells such as, for example, monocytes, connective-tissue macrophages, Langerhans cells, osteoclast cells, dendritic cells, microglial cells, neutrophils, eosinophils, basophils, mast cells, T lymphocytes, such as, for example, helper T cells, suppressor T cells, and killer T cells, B lymphocytes, such as, for example, IgM, IgG, IgA, IgE, killer cells, and stem cells and committed progenitors for the blood and immune systems. In a particularly preferred embodiment, the cells comprise circulating blood cells such as lymphocytes, neutrophils, monocytes, eosinophils, red blood cells, platelets, and basophils.

The cells can be from any biological sample obtained from the subject. For example, in some embodiments, the biological sample will be blood and therefore the mixture of cells will comprise circulating blood cells. The present invention therefore describes a method by which the cytoskeletal protein content of circulating blood cells is assessed. In some embodiments, the biological sample will be a biopsy sample of a selected tissue in the body. Non-limiting examples of tissues include tissues from the liver, lung, heart, breast, and muscle. In some embodiments, the selected tissue will be diseased, e.g., cancerous. In order to evaluate the effect of a biologically active agent on cellular cytoskeletal signature, a biologically active agent can be provided to the mixture of cells before the cytoskeletal protein is assessed. In some embodiments, the biologically active agent will be a stimulant or a depressant. In one aspect, the agent is a toxin, such as for example, a bacterial or viral toxin. In another aspect, the agent is a drug or a small molecule. In some embodiments, the agent is an enzyme regulator, an immune modulator or a chemotherapeutic agent.

In accordance with the present invention, the present methods can further comprise a step of comparing the content of cytoskeletal protein associated with one or more cell types with the content of cytoskeletal protein associated with corresponding cell types from a control. In some embodiments, additional cellular parameters such as the size and granularity of one or more cell types are determined. In one embodiment, the size and granularity is determined by measuring the forward scatter and side scatter of the cells and correlating forward scatter and side scatter to cell size and granularity. Additional cellular parameters in one or more cell types can also be compared to corresponding cellular parameters in a control.

One skilled in the art would appreciate that comparing the content of the cytoskeletal protein can be performed in a number of ways. For the purposes of this application comparing the content of the cytoskeletal protein includes, but is not limited to, comparing the content of the cytoskeletal protein of corresponding cell types of two or more samples, comparing the correlation of cytoskeletal protein content and cell size and/or cell granularity and/or other cellular parameters, comparing the ratio of cytoskeletal protein content of one cell type to the cytoskeletal protein content of another cell type, and comparing the standard deviation, skewness, kurtosis, or other features of the distribution of cytoskeletal protein content.

The present invention provides, inter alia, methods of profiling subjects based on the biophysical properties, including the cytoskeleton signature, of their cells.

Unless noted otherwise, any method of assessing the content of cytoskeletal protein in a cell, including, for example, measuring the polymerization state of a cytoskeletal protein and/or detecting fluorescence or other energy absorbed by, and/or emitted from, the cytoskeletal protein or a label attached to the cytoskeletal protein can be used in the present invention.

In some instances, it will be desirable to assess cytoskeletal content and/or other cellular parameters associated with live cells in order to assess live cytoskeletal protein contents or to monitor live cell responses to stimuli. In other instances, it will be desirable to assess cytoskeletal content in cells that have been stabilized, i.e., by fixation. The present invention includes both methods of assessing cytoskeletal protein in live and stabilized cell samples. Methods of measuring actin polymerization include, for example, fluorescence enhancement of pyrene conjugates, DNase inhibition assays, viscosity measurements, and spin-down assays. (Cooper et al., Methods in Enzymology, 1982, 182-211). For example, a plurality of cells obtained from a subject can be mixed with an amount of pyrene conjugated actin and polymerization can be measured with a fluorescence spectrophotometer. Fluorescence is enhanced with polymerization. In some embodiments, propidium iodide, which binds preferentially to double-stranded DNA, can be used to correlate cell cycle distribution with cytoskeletal protein response in each cellular subset. Due to its ability to intercalate into double-stranded DNA, propidium iodide can be used in ploidy analysis, hence cell cycle analysis (Krishan, J. Cell. Biol. 1975, 66:188-193). Similarly, propidium iodide or other agents can also be used to study apoptotic cell death and to correlate apoptosis with cytoskeleton changes as reflected in cytoskeletal protein measurements.

Cell cycles and apoptosis are non-limiting examples of cellular responses that can be correlated with cytoskeletal protein measurements according to methods of the present invention. One skilled in the art would appreciate that other cellular responses may also be studies and correlated with cytoskeletal measurements according to methods disclosed herein.

It has been discovered by the present inventor that in order to minimize the introduction of artifacts while assessing the content of cytoskeletal protein and thus cellular responses, it is preferable to minimize the handling of the cells and to mimic the in vivo cellular environment. Accordingly, the present invention provides methods of assessing the content of cytoskeletal protein that do not involve the purification of cellular subsets. By measuring the cytoskeletal protein in a sample that comprises a mixture of cells, it is also possible to measure cytoskeletal protein in a plurality of cell types simultaneously thereby providing information about cytoskeletal protein content in a plurality of cell types.

It has also been discovered by the present inventor that cytoskeletal protein is not always adequately preserved when stabilized at standard temperatures used for cell fixation. For example, it has been discovered that at a temperature of about 4 degrees Celsius, a common temperature for cell fixation, accurate measurement of the in vivo state of cellular actin contents is impaired. It had been heretofore unknown that cytoskeletal concentrations are extremely sensitive to temperature change. Accordingly, in some embodiments, the present invention provides methods of stabilizing mixtures of cells under conditions which better preserve cellular cytoskeletal protein. In some embodiments, conditions which better preserve cellular cytoskeletal protein are temperature levels that are in the vicinity of physiological temperature, for example, temperatures greater than about 25 degrees Celsius, preferably temperatures of about 30 degrees Celsius, more preferably temperatures of about 35 degrees or 37 degrees Celsius or higher. In one embodiment, the cells are stabilized at a temperature of from about 27 degrees to about 50 degrees Celsius. In another embodiment, the cells are stabilized at a temperature of from about 30 degrees to about 40 degrees Celsius, preferably at a temperature of about 37 degrees Celsius. According to some embodiments, selected reagents and solutions used in the present methods are pre-equilibrated to a temperature that better preserves cytoskeletal protein.

It has also been discovered that calcium ion concentration has an effect on the inducibility of cytoskeletal polymerization in cells. Accordingly the present invention provides methods of collecting a mixture of cells from a subject wherein the cells are collected using a non-chelating anticoagulant. It had heretofore been unknown that chelating agents have a distorting effect on cytoskeletal protein levels and interfere with the ability to accurately assess cytoskeletal protein content in a cell. Accordingly, in some embodiments, conditions which better preserve cellular cytoskeletal protein are those in which calcium concentrations of the cells have not been altered. In some embodiments, biological samples are collected in tubes containing one or more non-chelating anticoagulants, e.g., heparins or heparinoids. In some other embodiments, the tubes are maintained at or near the selected temperature.

In addition to the artifacts arising from non-physiological temperatures and altered calcium concentrations, other factors in purification processes can affect the accuracy of the cytoskeletal protein measurements, e.g., centrifugal forces. Research over the past few years has shown that cellular behaviors can be dramatically altered under different gravitational loadings. For example, when cells are exposed to lower gravitational loading (e.g., microgravity culture; Hashemi, FASEB J. 1999, 13: 2071-2082) or hyper gravity (e.g., centrifugation), their responses to stimulating agents are altered. Therefore, purification of specific cell types by centrifugation can have a significant impact on cellular skeletal protein contents or their polymerization state, which in turn affect cellular responses to stimuli. Accordingly, the present invention provides methods of minimizing the introduction of artifacts in assessing the responsiveness of cells by minimizing the manipulation following sample collection from donors. For example, in some methods of the present invention, the exposure of cells to high centrifugal forces prior to stabilization of cells is avoided, e.g., forces over 200 g. In some methods of the present invention, the exposure of cells to any centrifugal forces prior to stabilization of cells is avoided.

Methods according to embodiments of the invention can be used to simultaneously measure cytoskeletal protein contents in a plurality of cell types in a mixture of cells. In some embodiments, these methods are performed in a temperature range close to the normal physiological temperatures, e.g., about 30° C.-40° C., preferably around about 37° C., to avoid artifacts. Simultaneous measurements as used herein refer to measurements of cytoskeletal protein contents in several cell types in a mixture of cells without having to purify each cell type. As used herein the term “simultaneousness” does not mean chronologically at the same time. A plurality of cell types refers to at least two or more cell types.

In some embodiments of the present invention, before assessing the content of cytoskeletal protein associated with one or more cells, the cells are stabilized. Methods of stabilizing cells are known in the art and are thus not described herein in detail. Cells can be stabilized, for example, by cross-linking cellular protein, e.g., by fixation. Various chemicals including, for example, alcohol, formaldehyde, or glutaraldehyde, can be used to fix the cells. In some embodiments, the fixative solution will contain additional ingredients. For example, the fixative solution can also contain a membrane permeabilization agent, such as saponin or other surfactants/detergents. An exemplary fixative solution comprises about 3.7% formaldehyde and about 0.1% saponin in PBS.

The present invention provides methods for measuring cellular responses comprising a step of stabilizing a mixture of cells comprising one cell type or a plurality of cell types from a subject. Methods of collecting biological samples such as blood or other tissues comprising one cell type or a plurality of cell types are known and are thus not described herein in detail. In an exemplary embodiment of the present invention, an aliquot of blood sample is placed into each of a set of assay tubes. In some embodiments, the blood samples and the assay tubes have been pre-equilibrated to physiological temperature, e.g., about 37° C. The cells are then stabilized by providing a selected amount of a fixative solution to each assay tube. A fixative solution is any solution that fixes the cells. Typically, a fixative solution is a buffer solution (e.g., phosphate-buffered saline, PBS) comprising one or more cell fixation reagents (e.g., formaldehyde or glutaraldehyde). The assay tubes are then incubated at a selected temperature, e.g., from about 4 to about 50 degrees Celsius, preferably from about 30 to about 40 degrees Celsius, for a sufficient period of time in order to achieve stabilization of the cells. Stabilization and permeabilization can be achieved in multiple steps or in a single step. Any permeabilization solution can be used in the present methods. For example, in some embodiments, the permeabilization solution can comprises a surfactant (e.g., saponin or other surfactants/detergents—triton, alkyl glucosides, and the like.) in a buffer (e.g., PBS or other buffers). The solution can further comprise of, for example, additional ingredients such as a preservative or oxidation inhibitor (e.g., sodium azide). A permeabilization solution can comprise, for example, 0.1% saponin and 0.01% sodium azide in PBS.

In some embodiments of the present invention, during fixation of the sample, the sample is diluted in the fixative in order to improve efficiency of the fixation as well as to reduce fixation artifacts. For example, in some embodiments, the dilution ratio will be from about 1:1 to about 50:1 (e.g., 1:1, 2:1, 5:1, 10:1 or 20:1), preferably from about 10:1 to about 30:1.

The methods of the present invention are not limited to methods that include a step of assessing the content of cytoskeletal protein. The described methods of stabilizing a mixture of cells at temperatures of from about 27 degrees Celsius to about 50 degrees Celsius, preferably at temperatures of from about 30 degrees Celsius to about 40 degrees Celsius and more preferably at about 37 degrees Celsius can be performed on any cell sample. Accordingly, the present invention provides methods for preserving a cell comprising stabilizing a mixture of cells comprising one cell type or a plurality of cell types at temperatures of from about 27 degrees Celsius to about 50 degrees Celsius, preferably temperatures from about 30 degrees Celsius to about 40 degrees Celsius and more preferably temperatures of about 37 degrees Celsius. In some embodiments, the mixture of cells will be collected from a subject using a non-chelating anticoagulant. Additionally, in some embodiments, exposure of the cells to high centrifugal forces or even any centrifugation before stabilization will be avoided.

In some embodiments of the present invention, blood is collected at a remote site, stabilized at the remote site, and transferred to an appropriate facility for further analysis. In other embodiments, the blood is transferred before stabilization. In some embodiments, the mixture of cells will be cells from tissue culture and not from a particular subject.

After stabilization, the samples can, for example, be centrifuged at a selected centrifugal force for a suitable period of time to sediment the cells. The supernatants can be removed by any known method, for example by decanting, siphoning, suction, or filtration. In some embodiments a wash step is used to remove any excess fixative. In some embodiments, staining solution can then be added to each assay tube containing the sedimented cells in order to label cell types and/or cytoskeletal protein.

In some embodiments of the present invention, the cellular subsets or plurality of cell types are labeled with a cell-type specific reagent. A cell-type specific reagent as used herein refers to any reagent that can bind to and differentiate between specific cell types. In some embodiments, a cell type-specific reagent will comprise a reporter moiety, i.e., a detectable label, to facilitate its detection. Reporter molecules are known in the art and include, for example, fluorophores, chromophores, radiolabels, such as radioisotopes, and affinity ligands, such as, for example, biotin, glutathione, or an oligonucleotide that can be specifically detected by addition of a labeled reagent such as, for example, avidin/strepavidin, glutathione S-transferase, or a complementary oligonucleotide. An oligonucleotide affinity ligand can be a synthetic oligonucleotide or a naturally occurring oligonucleotide. It can be, for example, DNA (deoxyribonucleic acid), RNA (ribonucleic acid), or the like (e.g., peptide nucleic acid, PNA). An oligonucleotide should have a sufficient length such that the binding to its complementary oligonucleotide will be stable at the temperature used for the experiments; typically, 10-mers or longer. The particular reporter molecule or detectable group used is not a critical aspect of the invention. It can be any material having a detectable physical or chemical property. Thus, a reporter molecule or label is any composition detectable by, for example, spectroscopic, photochemical, biochemical, immunochemical, electrical, optical or chemical means. Additional examples include, magnetic beads, fluorescent dyes, enzymes, and colorimetric labels such as colloidal gold or colored glass or plastic beads. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the cytoskeletal protein or used to detect antibodies specifically reactive with the cytoskeletal protein.

In some embodiments of the present invention, a cell type-specific reagent can also comprise a binding agent that binds to cell-specific membrane proteins. For example, CD56 molecules are typically found on neural cells, tumors, and lymphocytes that mediate non-MHC-restricted cytotoxicity. Thus, a binding agent, such as for example, an antibody, that binds specifically or preferentially to CD56 can be used to specifically label this subpopulation of lymphocytes. Similarly, CD3 molecules are typically found on mature T lymphocytes (T cells) and these molecules associate with T-Cell receptors (TCR); hence, antibodies against CD3 can be used to label this population of T cells. CD14 is a glycolipid-anchored membrane glycoproteins expressed on cells of the myelomonocyte lineage, including monocytes, macrophages, and some granulocytes. Thus, antibodies against CD14 can be used to label these types of cells. A skilled practitioner will be able to choose a suitable binding agent for use in the present invention.

In some embodiments of the present invention, the reporter moiety will be a fluorescent molecule and the labeled cells will be detected using microscopy techniques or flow cytometry techniques, e.g., in some embodiments a fluorescence-activated cell sorter (FACS) will be used. Examples of fluorescent cell-specific labeling reagents include, for example, those sold by Beckon Dickinson and Company (Franklin Lakes, N.J.) under the trade names of perCP-CD3™ and APC-CD14™.

Cytoskeletal protein analysis does not require labeling of cell types. For example, the total cytoskeletal protein content in the mixture of cells can be assessed and provide information as to cellular responses, cytoskeletal signatures, biomolecular signatures, and the like.

In some embodiments, cytoskeletal protein is labeled. Cytoskeletal protein labeling solution as used herein refers to solution containing one or more reagents that can bind specifically to a certain type of skeletal protein as opposed to other molecules. For example, actin-labeling solution as used herein refers to a solution containing one or more reagents that can bind specifically or preferentially to actin molecules (G-actin or F-actin, or both), as opposed to other molecules.

Any reagent, including probes, that can preferentially bind to cytoskeletal protein such as, but not limited to, actin microfilaments, intermediate filaments, microtubules, spectrin, talin, vinculin, desmin, senaptin, vimentin, ezrin, moesin, filamin, phakinin, actinin, profilin, fibrin, keratin, myosin, dynein, and kinesin can be used in the present invention to assess the cellular content of cytoskeletal protein. Reagents that preferentially bind to actin molecules include, for example, anti-actin antibody, cytochalasin D, phalloidin, and phallacidin. Cytochalasin D binds to the plus ends of F-actin filaments and prevents further addition of G-actin. Phalloidin and phallacidin are cyclic peptides from the Death Cap fungus (Amanita pkalloides) that bind to and stabilize F-actin filaments. Reagents that preferentially bind to tubulin include, for example, anti-tubulin antibodies, paclitaxel, paclitaxel conjugates, and BODIOYP FL vinblastine. Reagents that preferentially bind to other cytosekeleton proteins include, for example, phosphoinositides and related products, anti-glial fibrillary acid protein antibody, anti-desmin antibody, anti-synapsin antibody, and endostatin protein.

Cytoskeletal protein binding reagents are typically coupled to a reporter moiety to facilitate their detection. A reporter moiety can include, for example, a fluorophore, a chromophore, a radio isotope, or an affinity ligand, such as, for example, biotin or an oligonucleotide that can be specifically detected by the addition of a labeled reagent, for example, avidin or the complementary oligonucleotide. Commonly used fluorophores can include, for example, NBD, 7-nitrobenz-2-oxa-1,3-diazol-4-yl; FITC, fluorocein isothiocyanate; and BODIPYTM, 4,4,-difluoro-3a,4a-diaza-s-indacene. A reagent that contains an actin-binding moiety and a reporter moiety will be referred to herein as an “actin probe”. Molecular Probes, Inc. (Eugene, Oreg.) offers various labeled phalloidin and phallacidin, including those under the trade names of BODIPY™-phalloidins and BODIPY™-phallacidins with different excitation and emission wavelengths. An exemplary F-actin labeling solution, e.g., F-actin probe, can be prepared by drying 30 ul of a methanol stock solution of BODIPY™-phallacidin, which has been prepared according to the instructions from the supplier, in a glass tube, followed by addition of the permeabilization solution as described above. The particular reporter molecule or detectable group used is not a critical aspect of the invention. It can be any material having a detectable physical or chemical property. Thus, a reporter molecule or label is any composition detectable by spectroscopic, photochemical, biochemical, immunochemical, electrical, optical or chemical means. Additional examples include, magnetic beads, fluorescent dyes, enzymes, and colorimetric labels such as colloidal gold or colored glass or plastic beads.

Cytoskeletal protein assessment does not require labeling of cytoskeletal protein in the cells. For example, cytoskeletal protein content can be determined by measuring the absorption profiles of the cells at various wavelengths of light.

In some embodiments, the cells can be suspended in the staining, i.e., labeling, solution by tapping, mixing, shaking, or the like, followed by a period of incubation time for labeling of the cytoskeletal protein to occur. A selected amount of a wash solution, for example, PBS containing 0.1% saponin and 0.01% sodium azide, can be added to each assay tube followed by centrifugation to sediment the cells. The supernatants are discarded, for example by decantation, and the cells re-suspended in a suitable amount storage solution.

Some embodiments of the present invention involve the step of assessing the content of cytoskeletal protein associated with a cell. Any method of assessing the content of cytoskeletal protein can be used in the present invention. The step of assessing the content of cytoskeletal protein can be performed at a remote location. In embodiments of the present invention wherein the cytoskeletal protein is labeled, assessing the content of the cytoskeletal protein can be as simple as detecting the label. Means of detecting labels are well known in the art. Thus, for example, where the label is a radioactive label, means for detecting can include a scintillation counter or photographic film as in autoradiography. Where the label is a fluorescent label, it can be detected by exciting the fluorochrome with the appropriate wavelength of light and detecting the resultant fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resultant reaction product. Colorimetric or chemiluminescent labels can be detected by simply observing the color associated with the label. Similarly, embodiments of the invention can be adapted to miniature assay formats (e.g., 96-wells plates, chips, and the like.). Furthermore, various steps as described above may be performed automatically by machines.

Methods known to those of skill in the art for detection of nucleic acids and proteins can be used, for example, PCR, northern and Southern blots, dot blots, nucleic acid arrays, western blots, immunoassays such as immunoprecipitation, ELISA, proteomics assays, and the like.

In some embodiments, the cell content is assessed by flow cytometry for the measurements of intracellular levels of cytoskeletal protein in cellular subsets. There are several commercially available flow cytometers, including FACS instruments, that can be used in these methods; they are not part of the invention and should not limit the present invention. One exemplary flow cytometer is sold by Becton Dickinson and Company (Franklin Lakes, N.J.) under the trade name of FACSCalibur™.

Additional biophysical cellular parameters can be assessed in addition to the content of cellular cytoskeletal protein. For example, an index of cell size and cell granularity can be assessed using the methods of the present invention. In some embodiments of the present invention, forward and side scatter data can be used as a proxy for cell size and granularity respectively. For example, when a laser hits the cell, the larger the cell the more photons of light it scatters. By measuring the light scattered on the side of a cell furthest from where the laser hits the cell, a measure of cell size can be obtained. Similarly, the more granular a cell the more light it will scatter 90 degrees to the incident laser beam (i.e. side scatter). A cell that has more dense granules will scatter more light to the side. Additional parameters, include, but are not limited to, cellular absorption, autofluorescence, cell count ratios (e.g., looking at CD4/CD8 cell ratios), and receptor count ratios on the cell surface.

As previously described, flow cytometry can be used to assess the cytoskeletal protein of the cell as well as to assess cell size and granularity. Methods of using flow cytometry to measure cellular biophysical parameters are known in the art and are thus not described herein in detail. In one embodiment, flow cytometry gating techniques are used to assess the biophysical cellular parameters. Flow cytometry can be used to gate on a plurality of cell types and obtain measurements from the different cellular subtypes. For example, in one embodiment of the present invention, flow cytometry is used to simultaneously assess the F-actin content of T-cells, monocytes and neutrophils. Three gates are defined to select for different cells. For example, neutrophils are selected based on the Side Scatter/Forward Scatter histogram; T-cells are selected based on the CD3 channel; and monocytes are selected based on the CD14 channel. The actin content of each cell type is then measured using the actin channel and the fluorescence level of each cell can be displayed on a corresponding histogram of each cell type. In this manner, the relative mean fluorescence associated with the F-actin content of each cell population is calculated using statistical analysis of the data. Other cellular sub-populations can readily be analyzed with this technique. For example, probes for CD4 and CD8 may be used to further differentiate the helper and cytotoxic T cells subgroups, respectively. Other cytoskeletal proteins can also be readily analyzed with this technique.

To improve measurement reliability, each cellular sample can be analyzed, for example, by flow cytometry in duplicate or triplicate. The average cytoskeletal protein fluorescence and the standard error of mean can then be calculated for each data point. For example, in one embodiment, an experiment with 6 donors over 4 time points during a 24 day period will consist of 4 blood collections per donor. In one example, 90 ul of blood from each blood sample is cultured in each of six assay tubes at 37° C. Three of the assay tubes receive a stimulation (for example, OKT3 to activate the T-cells) and the other three are used as control. That is, the experiment is performed in triplicate. After sample processing, each tube is analyzed by flow cytometry to generate one data point for each cellular subpopulation.

Using methods of the present invention, convenient measurements of cytoskeletal protein in or on the surface of various cells can be performed in a plurality of cells without having to first purify each cell. These methods make it possible to study cytoskeletal protein/differences in various cells as a function of time or among individuals. For example, it is possible to follow the cellular cytoskeletal protein contents and to monitor the inducible cellular cytoskeletal protein contents of each cell type for each test subject over time.

The present invention provides methods of identifying the cytoskeletal signature of cells, biomolecular signature of cells as well as cellular responses to agents that have an effect when provided to a cell, e.g., biologically active agent. Such agents, for example, can act as either stimulants or depressants. For use herein, a stimulant is any biologically active agent that produces a temporary or permanent increase in the functional activity or efficiency of an organism or any of its parts. For use herein, a depressant is any biologically active agent that produces a temporary or permanent decrease in the functional activity or efficiency of an organism or any of its parts. In some embodiments, the biologically active agent will be neither a stimulant nor a depressant but will have a measurable effect on the cytoskeletal or biomolecular signature of a cell.

In certain embodiments, the biologically active agent will be exogenously administered to the mixture of cells or plurality of cell types after the cells have been obtained from the subject. In some other embodiments, the subject will have been exposed to the agent or suspected to have been exposed to the agent before collection of the cells. In some embodiments, the mixture of cells will be cells from tissue culture and not from a particular subject. Any biologically active agent can be used in the present methods in order to, for example, measure the cellular effect of the agent or identify a cytoskeletal or biomolecular signature associated with the agent. Such agents include, but are not limited to, pathogens (i.e., bacterial or viral toxins), and small molecules (i.e., drugs, peptides, and the like). These include, for example, antigens, antibodies, superantigens, chemotatic agents, chemotatic peptides, enzyme regulators, immune modulators, chemotherapeutic agents, FMLP, N-formyl-methionyl-leucyl-phenylalanine; protein kinase C activator, phorbol myristate acetate, PMA; anti-TCR/CD3 mAb; lectins; lipopolysacharides. LPS; calcium ionophores, A23187 or the like; ligands, and the like.

Pathogens include, but are not limited to, bacteria including gram-positive and gram-negative bacteria, fungi, parasites, viruses, and other chemical and biological toxins.

Bacteria include, but are not limited to, bacteria from the following species Aerococcus, Enterococcus, Halococcus, Leuconostoc, Micrococcus, Mobiluncus, Moraxella catarrhalis, Neisseria (including N. gonorrheae and N. meningitidis), Pediococcus, Peptostreptococcus, Staphylococcus (including S. aureus, S. epidermidis, S. faecalis, and S. saprophyticus), Streptococcus (including S. pyogenes, S. agalactiae, S. bovis, S. pneumoniae, S. mutans, S. sanguis, S. equi, S. equinus, S. thermophilus, S. morbillorum, S. hansenii, S. pleomorphus, and S. parvulus), Veillonella; Acetobacter, Acinetobacter, Actinobacillus equuli, Aeromonas, Agrobacterium, Alcaligenes, Aquaspirillum, Arcanobacterium haemolyticum, Bacillus (including B. cereus and B. anthracis), Bacteroides (including B. fragilis), Bartonella, Bordetella (including B. pertussis), Brochothrix, Brucella, Burkholderia cepacia, Calymmatobacterium granulomatis, Campylobacter (including C. jejuni), Capnocytophaga, Caulobacter, Chromobacterium violaceum, Citrobacter, Clostridium species (including C. perfringens, C. tetani and C. difficile), Comamonas, Curtobacterium, Edwardsiella, Eikenella, Enterobacter, Erwinia, Erysipelothrix, Escherichia species (including E. coli), Flavobacterium (including F. meninosepticum), Francisella species (including F. tularensis), Fusobacterium (including F. nucleatum), Gardnerella (including G. vaginalis), Gluconobacter, Haemophilus (including H. influenzae and H. ducreyi), Hafnia, Helicobacter (including H. pylori), Herpetosiphon, Klebsiella species (including K. pneumoniae), Kluyvera, Lactobacillus, Legionella species (including L. pneumophila), Leptotrichia, Listeria species (including L. monocytogenes), Microbacterium, Morganella, Nitrobacter, Nitrosomonas, Pasteurella species (including P. multocida), Pectinatus, Porphyromonas gingivalis, Proteus species (including P. mirabilis), Providencia, Pseudomonas (including P. aeruginosa, P. mallei, P. pseudomallei and P. solanacearum), Rahnella, Renibacterium salmoninarum, Salmonella, Serratia, Shigella, Spirillum, Streptobacillus species (including S. moniliformis), Vibrio (including V. cholerae and V. vulnificus), Wolinella, Xanthobacter, Xenorhabdus, Yersinia species (including Y. pestis and Y. enterocolitica), Zanthomonas, Zymomonas, Crenothrix, Leptothrix Sphaerotilus, Beggiatoa, Gallionella, Sulfolobus, Thermothrix, Thiobacillus species (including T. ferroxidans), Thiomicrospira and Thiosphaera, Desulfobacter, Desulfobulbus, Desulfococcus, Desulfomonas, Desulfosarcina, Desulfotomaculum, Desulfovibrio, Desulfuromonas, Treponema species (including T. pallidum, T. pertenue, T. hyodysenteriae and T. denticola), Borrelia species (including B. burgdorferi and B. recurrentis), Leptospira and Serpulin, Acetobacterium, Actinomyces species (including A. israelii), Bifidobacterium, Brevibacterium, Corynebacterium species (including C. diphtheriae, C. insidiosum, C. michiganese, C. rathayi, C. sepedonicum, C. nebraskense), Dermatophilus, Eubacterium, Mycobacterium species (including M. tuberculosis and M. leprae), Nocardia, Propionibacterium, Rhodococcus, Streptomyces, Chondromyces, Cystobacter, Melittangium, Myxococcus, Nannocystis, Polyangium and Stigmatella, Mycoplasma species (including M. pneumoniae), Spiroplasma and Ureaplasma species (including U. urealyticum), Aegyptianella, Anaplasma, Chlamydia species (including C. pneumoniae, C. trachomatis and C. psittaci), Cowdria, Coxiella, Ehrlichia, Eperythrozoon, Haemobartonella, Neorickettsia, Rickettsia and Rickettsiella.

Fungi include but are not limited to, Acremonium, Aspergillus species (including A. flavus, A. niger, A. fumigatus, A. terreus, A. glaucus, and A. nidulans), Blastomyces species (including B. dermatitidis), Candida species (including C. albicans and C. parapsilosis), Ceratocystis, Chaetomium, Coccidioides species (including C. immitis), Cryptococcus species (including C. neoformans and C. laurenti) Epidermophyton, Fusarium species (including F. oxysporum and F. solani), Gongronella, Histoplasma species (including H. capsulatum), Acremonium, Honnonea, Lasiodiplodia theobromae, Malassezia furfur, Microsporum, Mycosphaerella fijiensis, Paracoccidiodes brasiliensis, Penicillium, Pneumocystis carinii, Pseudallescheria boydii, Pythium, Rhizoctonia, Rhodotorula, Saccharomyces, Sporothrix schenckii, Torula, Trichoderma, Trichophyton species (including T. mentagrophytes and T. rubrum) and Trichothecium.

Parasites include, but are not limited to, Acanthamoeba species, Ascaris lumbricoides, Babesia, Balamuthia, Balantidium, Blastocystis species including B. hominis, Chilomastix, Clonorchis sinensis, Cryptosporidium parvum, Cyclospora, Dientamoeba fragilis, Diphyllobothrium, Echinococcus, Endolimax, Entamoeba species (including E. histolytica), Enterobius species (including E. vermicularis), Giardia lamblia, hookworms (including Necator, Ancylostoma, and Unicinaria), Hymenolepsis, lodamoeba, Isospora, Leishmania, Mansonella, microsporidia, Microsporidium, Naegleria fowleri, Onchocerca, Plasmodium (including P. falciparum, P. vivax, P. ovale and P. malariae), Schistosoma (including S. haematobium and S. mansoni), Strongyloides species (including S. stercoralis), tapeworms (including Taenia species), Toxoplasma (including T. gondii), Trichinella (including T. spiralis), Trichomonas vaginalis, Trichuris species including T. trichiura, Trypanosoma, Dirofilaria, Brugia, Wuchereria, Vorticella, Eimeria species, Hexamita species and Histomonas meleagidis.

Chemical and biological toxins include any chemical or biological toxin including chemical or biological warfare agents. A chemical or biological warfare agent is any agent that might be employed because of its direct toxic effect on humans, animals, and plants. Accordingly, all chemical substances whether gaseous, liquid or solid, which are developed, produced, stockpiled, and used for hostile purposes and whose toxic effects are used to interfere with or destroy the normal functions of humans, plants, or animals in such a way as to lead to death, temporary incapacitation, or permanent injury are encompassed by the term chemical warfare agent. In some embodiments, the poisonous effects may occur immediately. In others, poisonous effects may be delayed. Chemical warfare agents may be delivered by any means known to deliver harmful or non-harmful agents, for example, by artillery, bombs, grenades, missiles, spraying devices, dumping devices, postal system, and the like.

Exposure to a specific class of chemical warfare agents, commonly referred to as organophosphorus agents, is recognizable using the methods of the present invention. Organophosphorus agents include the class of warfare agents known as nerve agents. Nerve agents include any organophosphate ester derivative of phosphoric acid that causes a disruption in the normal neurological function of a human, animal, or plant. Examples include VE (O-Ethyl-S-[2-(diethylamino)ethyl] ethylphosphonothioate), VG (O,O-Diethyl-S-[2-(diethylamino)ethyl] phosphorothioate), VM (O-Ethyl-S-[2-(diethylamino)ethyl]methylphosphonothioate), VX (O-Ethyl-S-[2(diisopropylamino)ethyl]methylphosphonothioate), cyclosarin, sarin, tabun, and soman.

The present invention provides methods of providing a biologically active agent (e.g., stimulant or depressant) to a mixture of cells before cell stabilization in order to, for example, measure the cellular effect of the agent or identify a cytoskeletal or biomolecular signature associated with the agent. In some embodiments, the mixture of cells is incubated in a suitable buffer, for example, Hanks' buffer, with a cellular stimulant or depressant. The reagent's stimulant or depressant effects can then be calculated from changes in the cytoskeletal signature or biomolecular signature of the cell. This method can be used to study biological samples that are exposed to a number of reagents specific for the various cell types.

In some embodiments, the present invention provides methods of characterizing the impact of a pathogen (or other biologically active agent) on the cytoskeletal signature of one cell type or a plurality of cell types. The signature profile of cell types to pathogens can be evaluated through measurements of cytoskeletal signature in one cell type or a plurality of cell types as well as cell size index and granularity index of the cells types before and after exposure to a pathogen. The impact of each pathogen on the cytoskeletal signature can be measured at increasing concentration of pathogen (increasing toxin concentration or multiplicity of infection) to obtain a dose response curve for each pathogen as well as a time-course of evolution of the cytoskeletal protein signature. Statistical analysis routines can be performed to develop predictive models for identification of pathogen exposure by analyzing the cytoskeletal or biomolecular signature of the cell types. In this manner, controls can be created for comparison with samples from a subject suspected of having a disease state. By comparing the cytoskeletal signatures from a subject sample to the cytoskeletal signature of a control, a skilled practitioner can determine if a subject has been exposed to a particular pathogen. For example, in one embodiment, a mixture of cells comprising one cell type or a plurality of cells types will be exposed to a pathogen, such as for example, salmonella typhimurium. The content of cytoskeletal protein in the one cell type or plurality of cell types before and after exposure to the pathogen will be assessed as well as in some embodiments, other cellular parameters such as cell granularity and cell size of the plurality of cell types. Based upon the cytoskeletal protein content and in some embodiments, cell size and granularity, the cellular cytoskeletal signature in response to salmonella typhimurium infection will be determined and will act as the control. In order to determine if a subject has been exposed to salmonella typhimurium, the content of cytoskeletal protein in a corresponding cell types from the subject will be assessed as well as in some embodiments, the cell size and granularity of the corresponding cell types. The cellular cytoskeletal signature from the subject will then be compared to that of the control to determine if the subject has been exposed to salmonella typhimurium.

In this manner, it is possible, for example, to assess the presence or absence of a disease state and other clinical parameters in a subject. A clinical parameter is not limited to the presence or absence of a disease state but can also include, for example, risk of disease, state of disease, severity of disease, class of disease, response to treatment of disease, i.e., whether a subject will be a negative responder, positive responder, or non-responder, and the like.

According to the present invention, a practitioner will be able to use the assessment of cytoskeletal protein content associated with a subject's cells to qualify the status of the subject with respect to the clinical parameter. In some embodiments, a certain cellular cytoskeletal signature and/or biomolecular signature will be indicative of a clinical parameter associated with a specific disease state. Accordingly, the present invention provides methods of correlating specific measurements of actin cytoskeleton in one or a plurality of cell types with specific clinical parameters.

The cytoskeletal protein profile of any pathogen or other biologically active agent can be determined using the present methods.

Methods of the invention are useful in comparing cytoskeletal signatures or biomolecular signatures of a large number of subjects. For example, some methods of the present invention include the step of assessing the content of cytoskeletal protein in one cell type or a plurality of cell types from each of a plurality of subjects belonging to a least two population groups differing with respect to at least one clinical parameter associated with a disease state and comparing the content of corresponding cytoskeletal protein in said one cell type or plurality of cell types from said groups to each other to create cytoskeletal protein profiles that are associated with different clinical parameters. In this manner, changes in the cytoskeletal signature or biomolecular signature of the cells can be determined.

Cytoskeletal contents in various cells from various samples can be readily compared, for example, by plotting on the same graph for each time point to determinate sample-to-sample variability. With a large number of samples, it is possible to establish “normal” distributions for each cellular subset as a baseline for analysis of data from a specific study—the baseline cytoskeletal signature or biomolecular signature. In other words, the “normal” distributions of cytoskeletal contents in each cellular subset can be used in other studies, for example, in patient diagnosis. In addition, the “normal” cytoskeletal protein contents in various cells can be used as cellular signatures for screening general populations for disease outbreaks, effects of chemical or biological warfare/terrorism, and the like.

Embodiments of the present invention which allow simultaneous measurements of cytoskeletal protein associated with blood cellular subsets provide unique tools for rapidly assessing the status of cells and their responsiveness using the cytoskeletal protein levels as the signature of each cellular subset. These methods can be applied, for example, in clinical research, patient diagnostics, and individualized therapy where an evaluation of the status of blood cells and their responsiveness to various agents are useful for diagnosis of disease and evaluation of treatment protocols. It is also valuable, for example, in situations where evaluation of responsiveness of cells is needed.

In one application, the present methods can be used for individualized drug therapy in order to select the most appropriate drug for treatment of a patient. Typically, when a patient presents a condition to a physician, the physician has a multitude of drugs that he can use for treatment of the condition. Typically, the physician will randomly select one of the drugs for therapy. If the drug is not a good fit, the patient will return to the physician and receive a second prescription for another drug in the hopes that the second drug will be a better fit than the first. Using the methods of the present invention, a blood sample can be taken from the patient at the first visit and the responsiveness, of the subject to a select group of drugs can be determined by identifying the cytoskeletal signature of the circulating blood cells in response to the different drugs. A certain signature will be indicative of a positive responder, negative responder, or a non-responder. In this manner, the efficacy of the drug, the optimal dosage concentrations, and/or the potential side effects of the drug can be assessed before the drug is provided to patient. The appropriate drug can then be provided in the first instance thereby leading to improved patient outcomes and reducing the overall cost of treatment. In some embodiments, additional cellular parameters, such as, for example, cell size and shape, will be identified to provide a biomolecular signature that is indicative of a positive responder, negative responder, or a non-responder.

Methods of the present invention can be used to reduce the risk associated with drug development by identifying unique signatures associated with adverse side effects thereby enabling market introduction of otherwise failing drugs and reducing the overall cost of drug development. For example, during phase III clinical trial, if a drug is effective in 70% of patients but causes unacceptable side effects in the remaining 30%, it is highly unlikely that the drug will come to the market. Using the present methods, it is possible to identify the patients that will be positive responders, negative responders, or non-responders by their cellular signatures. Before prescribing the drug to a patient population, the patients will be screened to determine how they will respond to the drug, i.e. a blood or tissue sample will be obtained from the patient and the cytoskeletal profile of the cells will be assessed. Those that have cytoskeletal profiles that match the cytoskeletal profiles of patients that didn't respond well to the drug will be advised to take an alternative drug. Alternatively, patients that have cytoskeletal protein profiles indicating that they will be positive responders will be prescribed the drug. In some embodiments, additional cellular parameters, such as, for example, cell size and shape, will be identified and the patient's biomolecular signatures will be identified and subsequently matched.

The methods of the present invention can be used in drug discovery. The effect of a select compound on cellular signatures can be assessed and it can thereby be determined whether the drug will be effective in treating a condition or disease state.

The present methods are particularly useful for diagnosing conditions, evaluating whether certain drugs will have a desired effect, and determining prognoses. Immune-related diseases such as, for example, allergies; autoimmune diseases such as, for example, arthritis and lupus; immune related syndromes such as, for example, Wiskott Aldrich; cancers such as for example, leukemia; and multiple sclerosis are only a small subset of the diseases detectable by the present methods. Other exemplary diseases include, but are not limited to, for example, stroke, nephritis, renal fibrosis, chronic obstructive pulmonary disease, restenosis, renovascular disease, organ transplant rejection; diseases associated with abnormal angiogenesis; insulin resistance; vascular inflammation; cerebrovascular diseases; hypertension; respiratory diseases, such as asthma; heart failure; arrhythmia; angina; atherosclerosis; kidney failure; peripheral vascular disease; peripheral arterial disease; acute vascular syndromes; microvascular diseases; hypertension; Type I and II diabetes and related diseases; hyperglycemia; hyperinsulinemia; coronary heart disease; bacterial disease and viral disease, such as AIDS. By assessing the evolution of the cytoskeletal protein signature or biomolecular signature at different times during disease progression, the stage of disease can be determined as well as the likely prognosis.

The present methods can be used to create an inflammation index that categorizes inflammation at different stages by measuring cytoskeletal protein levels or biomolecular signatures at different stages of inflammation. The present methods can also be used to detect hormonal changes in the body by associating certain hormonal changes to cytoskeletal protein signatures and/or biomolecular signature. In this manner, early assessment of diseases can be made by detecting internal changes that precede a disease.

Donor-recipient compatibility for transplants can be assessed using the present methods. Cytoskeletal protein measurements can be taken pre-transplant to assess compatibility and reduce the risk of rejection. For example, a tissue sample can be obtained from the donor and a blood sample from the recipient. By determining the cytoskeletal or biomolecular signature of the recipient's cells, e.g., T cells, after contact with the tissue sample, it can be determined how the recipient will respond to the transplant. Cytoskeletal protein measurements can be taken post-transplant to detect early rejection or acceptance. The present methods can also be used to optimize immunosuppressant therapy by monitoring the cytoskeletal protein signatures or biomolecular signatures of immune cells.

The extent of radiation exposure can be assessed using the present methods. Certain cytoskeletal protein signatures or biomolecular signatures will be indicative of different levels of radiation exposure and cellular damage.

The present methods can be used for early detection of cancer as well as for the optimization of treatment protocols and analysis of biopsy samples. The present methods can also be used to optimize chemotherapy through assessment of the cytoskeletal or biomolecular signature of the patient.

The present invention also provides methods of screening blood samples, i.e., for blood blanks, in order to identify blood that is not fit for donation. Vaccine development and validation is included within the scope of the present invention. The present methods can be used to identify adverse reactions to vaccines and screen for adverse reactions prior to vaccination. For example, a child can be screened for a potential adverse reaction to a panel of vaccines prior to vaccination. If a negative reaction is detected, a second screen can be performed to determine which of the vaccines should not be administered. In addition, tests can be developed to determine the effectiveness of a vaccine in an individual, i.e., to determine whether the vaccine generated an immune readiness. The longevity of a vaccine in an individual can also be assessed.

The present methods can be used to screen the food supply for disease, e.g., evidence of bacterial infection or mad cow disease as well as for animal heath, e.g., rabies. The present methods can be used to assess aging in a subject by correlating cytoskeletal protein signatures or biomolecular signatures in certain cell types with aging.

The present invention provides methods for classifying cells and generating classification systems for classifying cells. In accordance with some embodiments, the method comprises a step of providing a learning set comprising a plurality of data objects. Each data object represents a biomolecular signature for which clinical data has been developed. The clinical data included in the data object includes biophysical cellular parameters such as, for example, content of cytoskeletal protein, cell size, cell shape and the like. Each cell sample is classified into one of at least two different clinical parameter classes. For example, the clinical parameters could include presence or absence of disease, risk of disease, stage of disease, response to treatment of disease or class of disease.

In some embodiments, the method can further comprise a step of training a classification algorithm with the learning set. Classification models can be formed using any suitable statistical classification (or “learning”) method that attempts to segregate bodies of data into classes based on objective parameters present in the data. Classification methods can be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, each data object includes data indicating the clinical parameter class to which the sample belongs. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In other embodiments, the classification models that are created can be formed using unsupervised learning methods. Unsupervised classification attempts to learn classifications based on similarities in the training data set. In this case, the data representing the class to which the sample belongs is not included in the data object representing that subject, or such data is not used in the analysis. Unsupervised learning methods include cluster analyses. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application 2003 0004402 A1 (Hitt et al., “Process for discriminating between biological states based on hidden patterns from biological data”), and U.S. Patent Application 2003 0055615 A1 (Zhang and Zhang, “Systems and methods for processing biological expression data”).

Thus classification model can be generated that classify a sample into one of the classification groups. The classification models can be used to classify an unknown sample into one of the groups.

The present invention also provides methods for maintaining a cytoskeletal protein signature or biomolecular signature registry system or database. Such a system can be managed using bioinformatics. Bioinformatics is the study and application of computer and statistical techniques to the management of biological information. Thus, in one embodiment, the present invention provides a method for populating a database for further medical characterization. For example, a database can be populated with the cytoskeletal protein signatures in a plurality of cell types that have been exposed to agents that act as, for example, stimulants, depressants, pathogens, bacterial and viral toxins, chemical warfare agents and the like. This information can be used for comparative purposes as a control. Once a database of sufficient size has been generated, clinical parameters can be determined by comparing cytoskeletal protein measurements and other cellular parameters in a plurality of cell types to corresponding cytoskeletal protein measurements and other cellular parameters in the controls.

In another embodiment, the present invention also provides an apparatus for automating the methods of the present invention, the apparatus comprising a computer and a software system capable of analyzing biomolecular signatures. The data is inputted in computer-readable form and stored in computer-retrievable format. The present invention also provides computer-readable medium encoded with a data set comprising cellular profiles of cells that have been exposed to agents that act as stimulants or depressants, pathogens, bacterial and viral toxins, chemical warfare agents and the like. The information in the data set can be used for comparison purposes.

The methods described herein for quantifying cellular cytoskeletal protein and other cellular parameters provides information which can be correlated with pathological conditions, predisposition to disease, therapeutic monitoring, risk stratification, among others. Although the data generated from the methods of this invention is suited for manual review and analysis, in a preferred embodiment, data processing using high-speed computers is utilized.

The invention also provides for the storage and retrieval of a collection of profiles and comparisons in a computer data storage apparatus, which can include, for example, magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays.

This invention also preferably provides a magnetic disk, such as an IBM-compatible (DOS, Windows, Windows 95/98/2000, Windows NT, OS/2, etc.) or other format, e.g., Linux, SunOS, Solaris, AIX, SCO, Unix, VMS, MV, Mactinosh etc., floppy diskette or hard (fixed, Winchester) disk drive, comprising a bit pattern encoding data collected from the methods of the present invention in a file format suitable for retrievable and processing in a computerized comparison or relative quantification method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device comprises a pattern of magnetic domains and/or charge domains comprising a bit pattern encoding data acquired from the methods of the invention.

The invention also provides a method for transmitting data that includes generating an electronic signal on an electronic communications device, such as a modem, ISDN terminal adapter, DSL, cable modem, ATM switch, or the like, wherein the signal includes (in native or encrypted format) a bit pattern encoding data collected using the methods of the present invention.

In one embodiment, the invention provides a computer system for performing methods of the present invention. A central processor is preferably initialized to load and execute the computer program for alignment and/or comparison of results. Data is entered into the central processor via an I/O device. Execution of the computer program results in the central processor retrieving the data from the data file. The target data or record and the computer program can be transferred to secondary memory, which is typically random access memory. For example, a central processor can be a conventional computer; a program can be a commercial or public domain molecular biology software package; a data file can be an optical or magnetic disk, a data server, or a memory device; an I/O device can be a terminal comprising a video display and a keyboard, a modem, an ISDN terminal adapter, an Ethernet port, a punched card reader, a magnetic strip reader, or other suitable I/O device.

The invention also provides the use of a computer system, such as that described above, which comprises, for example: (1) a computer; (2) a stored bit pattern encoding a collection of measurements obtained by the methods of the present invention, which may be stored in the computer; (3) a comparison control; and (4) a program for comparison.

This invention also provides kits for assessing cytoskeletal protein and screening cellular samples for clinical parameters. Such kits are useful, for example, for diagnostic or prognostic tests. Kits can include a solution for stabilizing cells, i.e., a fixative solution, labeling reagents for labeling cell types and/or cytoskeletal protein. The kit can also include instructions to detect and quantify the cytoskeletal protein in a sample, as well as instructions to correlate the amount of cytoskeletal protein detected with diagnostic and prognostic methods and/or screening methods according to the present invention.

All publications and patent documents cited above are hereby incorporated by reference in their entirety for all purposes to the same extent as if each were so individually denoted.

The below examples are non-limiting and for illustrating the present invention. Alternatives and variations of the below examples within the scope of the present invention as per the below claims may be carried out by a person skilled in the art.

### EXAMPLES

### Example 1

### Measuring the Impact of Pathogens on the Actin Signature Using T-Cells, Monocytes, and Neutrophils

Materials: Chemical toxins such as hexachlorobenzene, chloropicrin, and cyclosporin A will be purchased from Sigma-Aldrich Chemical Co. Appropriate CDC-approved suppliers of ricin toxin, purified bacterial toxins, and influenza A virus stocks will be utilized. Human leukocyte cell lines Jurkat(T-cells), U937(monocytes), and HL60(neutrophils), and all bacterial cultures will be obtained from ATCC or other CDC approved source. Appropriate bacterial culture media will be purchased from Difco Co., and equipment for anaerobic culture of Clostridium sp will be obtained from Carolina Biologicals, Inc. Human cell culture media appropriate for each leukocyte type will be obtained from GIBCO-BRL Laboratories. Cell culture and bacterial culture supplies will be obtained from Fisher Scientific Co. Blood samples will be obtained from healthy donors.

The impact of specific pathogens on the cytoskeletal actin signature of whole blood cultures will be assessed at increasing concentration of pathogen (toxin concentration or multiplicity of infection) to obtain dose response curve for each pathogen. (Viability of cells will be determined for each culture condition by trypan blue exclusion protocol.)

Experiments will be carried out to determine dose-response curves of the toxic effects of each pathogen in whole blood cultures. From stock solutions of purified toxins aliquots representing increasing dose level in the pg/ml to ug/ml range will be added to blood cultures in order to determine the effects of increasing dose levels on the actin signature.

Suspensions of live bacteria, parasite, and virus of known particle concentrations aliquots representing increasing MOI in the range 10-10⁵ infectious units/ml will be added to blood cultures, and signatures will be measured after 60 minutes of pathogen exposure at 37° C.

The time-course of response of whole blood cultures to specific pathogens will be determined as measured by the evolution of the actin Signature. (Viability of cells will be determined for each culture condition by trypan blue exclusion protocol.)

Purified chemical and bacterial toxins will be put into stock solutions at known concentration, and from these stocks the toxins will be added to blood cultures. Blood cultures will be exposed to various concentration of toxin, and aliquots of the leukocyte cultures will be removed at periodic intervals (5 minutes to 4 hours) for measurement of the actin signature. The evolution of signatures will be analyzed over extended period of time (up to 2 weeks) by performing similar experiments using leukocyte cell lines.

Suspensions of known particle concentrations of live bacteria, parasite, and virus materials will be prepared, and from these stocks the live organisms or infectious virus particles will be added to whole blood cultures over a wide range of multiplicities of infection (MOI) such as 10-10⁴ infectious particles/ml. Aliquots of the leukocyte cultures will be removed at periodic intervals (5 minutes to 4 hours) for determination of the actin signature. The evolution of signatures will be analyzed over extended period of time (up to 2 weeks) by performing similar experiments using leukocyte cell lines.

The data generated by this technology consists of vectors in a multidimensional parameter space of non-negative real numbers. For each cell type, 6 parameters corresponding to the F-actin level, Forward Scatter (a measure of cell size), and Side Scatter (a measure of granularity), before and after receptor-mediated stimulation will be measured. Stimulation of cells with an activator cocktail containing LPS, FMLP, and OKT3 will be used to assess the ability of the cells to respond to stimulation. The sensitivity and resolution of the technology will be improved by increasing the number of cell types analyzed. Each additional cell type will provide 6 new signature parameters in addition to the 18 parameters currently used. The enhanced resolution of the signature will improve the ability to resolve signatures from different pathogens.

The 18-dimensional vector currently generated defines the signature of a blood sample. The basic premise of the technology is that specific pathogens cause unique shifts in these signatures in the 18-dimensional space, and that pathogens (or classes of pathogens that act by similar mechanism of action) can be identified by classifying signatures using statistical analysis and feature recognition.

Predictive classification models will be developed which maximize the probability of correctly classifying an unknown sample. SAS statistical analysis software (SAS Institute, 1999) will be the primary tool used for statistical analysis, graphing and reporting. Data exploration using scatter plots, summary statistics, and other descriptive tools will be used to understand the data and characterize any systematic variation caused by donor characteristics such as age, gender, and race.

The statistical model used in this investigation is the Multinomial Logistic Regression (MLR) (Hosmer et. al., (2000) Applied Logistic Regression, Wiley.) implemented by the SAS procedures GENMOD or LOGISTIC which allows the representation and analysis of complex models incorporating discrete as well as continuous independent variables. Repeated measurements on the same sample will be used to account for repeated sample variability, e.g., small temperature effects that will be accounted for by use of General Estimating Equations which are implemented by the “REPEATED” statement of GENMOD.

An efficient MLR model will be developed which will reduce the probability of misclassification and maximize the probability of correct classification. The strategy used for this purpose is to first assemble training sets of data from healthy donor blood samples exposed to specific pathogens. The MLR model will be developed using GENMOD or LOGISTIC and the training data sets. The resultant “df” will be tested against calibration data (signatures) whose classification in terms of normal, or exposed to a specific pathogen, is known. Performance of the model against the new signature will be used to further refine the model through an iterative process until the model gives acceptable results in terms of correct classification with respect to the calibration data set. A number of important variables such as pathogen concentration and exposure time will be fully examined.

The best subset of parameter features that have predictive power for recognition of exposure to pathogens will thus be determined.

In the presence of secondary infections, additional analysis will be required to resolve complex signatures for identification of pathogen exposure. For example, secondary bacterial infection following influenza exposure will result in a signature that is more complex than infection by one pathogen alone. These complex signatures can be resolved by mathematical modeling of signatures from singular infections. Mathematical models are routinely used to resolve complex signals in physical systems, and these models will be employed to develop methods for resolving signatures from secondary infection. For example, signatures from secondary infections can be expressed as a function of singular infection signatures, and regression analysis may be used to identify the infection agents. Co-infection studies will be performed using NIAID priority pathogens to develop models for resolving complex signatures resulting from multiple infections.

Co-Infection Studies for Resolution of Complex Signatures

Whole blood cultures will be infected with combinations of toxins and pathogens to obtain complex signatures similar to those that will be present in secondary infections. Combinatorial mathematical models will be used to evaluate actin signatures for identifying features that are representative of multiple exposures and identification of agents. Blind studies will be performed to assess the reliability of the models for accurate detection of exposure to multiple agents.

The same techniques will be performed for other cytoskeletal protein for example, intermediate filaments, microtubules, spectrin, talin, vinculin, desmin, senaptin, vimentin, ezrin, moesin, filamin, phakinin, actinin, profilin, fibrin, keratin, myosin, dynein, and kinesin.

1. A method for measuring a cellular response comprising:
i. stabilizing a mixture of cells comprising a plurality of cell types;
ii. labeling two or more cell types from the mixture using cell type-specific reagent; and
iii. assessing the content of cytoskeletal protein associated with the two or more cell types.

2. The method of claim 1 further comprising a step of:
iv. comparing the content of the cytoskeletal protein associated with the two or more cell types with the content of cytoskeletal protein associated with corresponding cell types from a control.

3. The method of claim 1 further comprising a step of determining the size and granularity of the two or more cell types.

4. The method of claim 3 further comprising a step of comparing the content of the cytoskeletal protein associated with the two or more cell types, the cell size, and the cell granularity of the two or more cell types with a content of cytoskeletal protein, cell size, and cell granularity in corresponding cell types from a control.

5. The method of claim 1 wherein the two or more cell types comprise at least one of an immune cell.

6. The method of claim 1 wherein the two or more cell types comprise at least one of a lymphocyte, neutrophil, monocyte, eosinophil, erythrocyte, platelet, or basophil.

7. The method of claim 1 wherein the cytoskeletal protein is F-actin.

8. The method of claim 1 wherein the mixture of cells is collected using a non-chelating anticoagulant.

9. The method of claim 1 wherein the cells are stabilized at a temperature of from about 27 to about 50 degrees Celsius.

10. The method of claim 1 wherein the cells are stabilized at physiological temperature.

11. The method of claim 1 wherein assessing the content of the cytoskeletal protein is performed using a flow cytometer.

12. The method of claim 1 further comprising the step of labeling cytoskeletal protein associated with the two or more cell types.

13. The method of claim 12 wherein assessing the content of the cytoskeletal protein is performed by microscopy.

14. The method of claim 1 wherein the cells are stabilized by fixation.

15. The method of claim 1 further comprising a step of providing a biologically active agent to the mixture of cells before stabilizing the cells.

16. The method of claim 15 wherein the biologically active agent is a stimulant or a depressant.

17. The method of claim 15 wherein the agent is a toxin.

18. The method of claim 15 wherein the agent is a bacterial or viral toxin.

19. The method of claim 15 wherein the agent is a drug or a small molecule.

20. The method of claim 19 wherein the agent is an enzyme regulator, immune modulator, or chemotherapeutic agent.

21. A method for measuring a cellular response comprising:
i. assessing the content of cytoskeletal protein associated with a plurality of cell types; and
ii. comparing the content of the cytoskeletal protein associated with said plurality of cell types to the content of corresponding cytoskeletal protein associated with corresponding cell types from a control.

22. The method of claim 21 further comprising a step of determining the size and granularity of the multiple cell types.

23. The method of claim 22 further comprising a step of comparing the content of the cytoskeletal protein associated with the plurality of cell types, the cell size, and the cell granularity of the plurality of cell types with a content of cytoskeletal protein, cell size, and cell granularity in corresponding cell types from a control.

24. The method of claim 21 wherein the plurality of cell types comprise immune cells.

25. The method of claim 21 wherein the plurality of cell types comprise at least one of a lymphocyte, neutrophil, monocyte, eosinophil, erythrocyte, platelet, or basophil.

26. The method of claim 21 wherein the cytoskeletal protein is F-actin.

27. The method of claim 21 wherein assessing the content of the cytoskeletal protein is performed using a flow cytometer.

28. The method of claim 21 further comprising a step of providing a biologically active agent to the plurality of cell types before assessing the content of cytoskeletal protein.

29. The method of claim 28 wherein the biologically active agent is a stimulant or a depressant.

30. The method of claim 28 wherein the agent is a toxin.

31. The method of claim 28 wherein the agent is a bacterial or viral toxin.

32. The method of claim 15 wherein the agent is a drug or a small molecule.

33. The method of claim 32 wherein the agent is an enzyme regulator, immune modulator, or chemotherapeutic agent.

34. A method for measuring a cellular response comprising:
i. stabilizing a mixture of cells comprising a plurality of cell types; and
ii. assessing the content of cytoskeletal protein associated with two or more cell types.

35. A method for measuring a cellular response comprising:
i. stabilizing a mixture of cells comprising one cell type or a plurality of cell types at a temperature of from about 27 to about 50 degrees Celsius; and
ii. assessing the content of cytoskeletal protein associated with the one or more cell types.

36. The method of claim 35 wherein the temperature is from about 30 to about 40 degrees Celsius.

37. A method for identifying a cytoskeletal signature comprising the step of:
i. assessing the content of cytoskeletal protein associated with a plurality of cell types.

38. The method of claim 37 further comprising the step of
ii. comparing the content of the cytoskeletal protein associated with said plurality of cell types to the content of corresponding cytoskeletal protein associated with corresponding cell types from a control.

39. The method of claim 37 further comprising a step of determining the size and granularity of the plurality of cell types.

40. The method of claim 39 further comprising a step of comparing the content of the cytoskeletal protein associated with the plurality of cell types, the cell size, and the cell granularity of the plurality of cell types with a content of cytoskeletal protein, cell size, and cell granularity in corresponding cell types from a control.

41. The method of claim 37 wherein the plurality of cell types comprise at least one of a lymphocyte, neutrophil, monocyte, eosinophil, erythrocyte, platelet, or basophil.

42. The method of claim 37 wherein the plurality of cell types comprise immune cells.

43. The method of claim 37 wherein the cytoskeletal protein is F-actin.

44. The method of claim 37 wherein assessing the content of the cytoskeletal protein is performed using a flow cytometer.

45. The method of claim 37 further comprising a step of providing a biologically active agent to the plurality of cell types before assessing the content of cytoskeletal protein.

46. The method of claim 45 wherein the biologically active agent is a stimulant or a depressant.

47. The method of claim 45 wherein the agent is a toxin.

48. The method of claim 45 wherein the agent is a bacterial or viral toxin.

49. The method of claim 15 wherein the agent is a drug or a small molecule.

50. The method of claim 49 wherein the agent is an enzyme regulator, immune modulator, or chemotherapeutic agent.

51. A method for assessing the presence or absence of a disease state in a subject comprising:
i. assessing the content of cytoskeletal protein associated with a plurality of cell types from the subject;
ii. correlating the content with the presence or absence of a disease state in the subject.

52. The method of claim 51 wherein said correlating step is performed by comparing the content of cytoskeletal protein associated with said plurality of cell types to the content of corresponding cytoskeletal protein associated with corresponding cell types from a control.

53. The method of claim 51 further comprising a step of determining the size and granularity of the plurality of cell types.

54. The method of claim 53 wherein said correlating step is performed by comparing the content of the cytoskeletal protein associated with the plurality of cell types, the cell size and the cell granularity of the plurality of cell types with a content of cytoskeletal protein, cell size, and cell granularity in corresponding cell types from a control.

55. The method of claim 51 wherein the plurality of cell types comprise at least one of a lymphocyte, neutrophil, monocyte, eosinophil, erythrocyte, platelet, or basophil.

56. The method of claim 51 wherein the plurality of cell types comprise immune cells.

57. The method of claim 51 wherein the cytoskeletal protein is F-actin.

58. The method of claim 51 wherein assessing the content of the cytoskeletal protein is performed using a flow cytometer.

59. The method of claim 51 wherein the disease state is bacterial infection.

60. The method of claim 51 wherein the disease state is viral infection.

61. The method of claim 51 wherein the disease state is cancer.

62. The method of claim 51 wherein the disease state is exposure to biological or chemical agent.

63. A method for measuring a clinical parameter in a subject comprising:
i. assessing the content of cytoskeletal protein associated with a plurality of cell types from each of a plurality of subjects belonging to a least two population groups differing with respect to at least one clinical parameter associated with a disease state;
ii. comparing the content of corresponding cytoskeletal protein associated with said plurality of cell types from said groups to each other to create cytoskeletal protein profiles that are associated with the clinical parameter.

64. A method for determining a response profile to a drug comprising
i. assessing the content of cytoskeletal protein associated with a plurality of cell types that have been exposed to the drug; and
ii. correlating the content of cytoskeletal protein with a probability of being a positive responder, negative responder, or non-responder to therapy with said drug.

65. The method of claim 64 wherein said correlating step is performed by comparing the content of cytoskeletal protein associated with said plurality of cell types to the content of corresponding cytoskeletal protein in corresponding cell types from a control.

66. The method of claim 64 further comprising a step of determining the size and granularity of the plurality of cell types.

67. The method of claim 66 wherein said correlating step is performed by comparing the content of the cytoskeletal protein associated with the plurality of cell types, the cell size and the cell granularity of the plurality of cell types with a content of cytoskeletal protein, cell size, and cell granularity in corresponding cell types from a control.

68. A method for monitoring the progression of a disease state in a subject comprising:
i. assessing the content of cytoskeletal protein associated with a plurality of cell types from the subject;
ii. correlating the content of cytoskeletal protein with progression of the disease state in the subject.

69. The method of claim 68 wherein said correlating step is performed by comparing the content of cytoskeletal protein associated with said plurality of cell types to the content of corresponding cytoskeletal protein in corresponding cell types from a control.

70. The method of claim 68 further comprising a step of determining the size and granularity of the plurality of cell types.

71. The method of claim 70 wherein said correlating step is performed by comparing the content of the cytoskeletal protein associated with the plurality of cell types, the cell size and the cell granularity of the plurality of cell types with a content of cytoskeletal protein, cell size, and cell granularity in corresponding cell types from a control.

72. The method of claim 68 further comprising a step of providing a biologically active agent to the plurality of cell types before assessing the content of cytoskeletal protein.

73. A method for determining donor-recipient compatibility for transplant therapy comprising:
i. assessing the content of cytoskeletal protein associated with a plurality of cell types from the recipient;
ii. correlating the content of cytoskeletal protein with compatibility to the transplant.

74. The method of claim 73 further comprising a step of determining the size and granularity of the plurality of cell types.

75. A method of generating a classification system for classifying a cell sample:
i. providing a learning set comprising a plurality of data objects, wherein each data object comprises data representing measurements of cytoskeletal protein in sample, and wherein the samples are classified according to at least two different clinical parameters; and
ii. generating a classification model, wherein the classification model classifies a cell sample as indicative of a clinical parameter, indication, or condition.

76. A method for measuring the content of cytoskeletal protein comprising:
i. stabilizing a mixture of cells comprising a plurality of cell types; and
ii. assessing the content of cytoskeletal protein associated with two or more of the cell types.

77. A method for preserving a cell comprising stabilizing a mixture of cells comprising one cell type or a plurality of cell types at a temperature of from about 27 to about 50 degrees Celsius.

78. The method of claim 77, wherein the temperature is from about 30 to about 40 degrees Celsius.
